(19) United States
(12) Patent Application Publication
(10) Pub. No.: US 2014/0112882 A1
(43) Pub. Date: Apr. 24, 2014

(54) Methods to treat pancreatic inflammation and associated lung injury through regulation of pancreatic interleukin-22 expression

(71) Applicants: Aida Habtezion, Sunnyvale, CA (US); Jing Xue, Sunnyvale, CA (US)

(72) Inventors: Aida Habtezion, Sunnyvale, CA (US); Jing Xue, Sunnyvale, CA (US)

(73) Assignee: The Board of Trustees of the Leland Stanford Junior University, Palo Alto, CA (US)

(21) Appl. No.: 14/048,889

(22) Filed: Oct. 8, 2013

Related U.S. Application Data
(60) Provisional application No. 61/711,060, filed on Oct. 8, 2012.
(60) Provisional application No. 61/721,317, filed on Nov. 1, 2012.

(51) Int. Cl. A61K 38/20; A61K 31/655; A61K 31/4025

(52) U.S. Cl. CPC A61K 38/20; A61K 31/4025; A61K 31/655
USPC 424/85.2; 514/422; 514/150

(57) ABSTRACT

Methods for use of a composition comprising agents that increase pancreatic interleukin-22 production in the treatment of pancreatic inflammatory disorders including pancreatitis-associated acute lung injury.

### CROSS-REFERENCE TO RELATED APPLICATION

This application claims priority and other benefits from U.S. Provisional Patent Application Ser. No. 61/711,060 filed Oct. 8, 2012, and Ser. No. 61/721,317 filed Nov. 1, 2012, both entitled “Methods to treat pancreactic inflammation and associated lung injury through regulation of pancreatic interleukin-22 expression”. Their entire contents are specifically incorporated herein by reference.

### STATEMENT OF GOVERNMENT SUPPORT

This invention was made with Government support under contracts DK56339 and DK092421 awarded by the National Institutes of Health. The Government has certain rights in this invention.

### TECHNICAL FIELD OF THE INVENTION

Provided herein are methods for attenuating pancreatic inflammation and pancreatitis-associated lung injury by administration of regulators of pancreatic IL-22 expression.

### BACKGROUND

Acute pancreatitis is thought to develop from an injury to the pancreatic acini, through leakage or inappropriate activation of pancreatic enzymes. In many cases, acute pancreatitis is a mild and short-lived inflammation which resolves spontaneously and without any detrimental consequences to the pancreatic tissues. However, in about 15-20% of all cases, acute pancreatitis manifests itself with an extensive pancreatic inflammation that is accompanied by necrosis of pancreatic tissue and subsequent organ failure which concerns many organs beyond the pancreas (Werner et al., 2003; Gaisano & Gorelick, 2009). Cases of acute pancreatitis that follow a severe course are associated with significant morbidity, since the initial pancreatic inflammation is followed by a systemic inflammatory response that progresses to sepsis, multiple organ dysfunction and possibly death (Gravante et al., 2009). Aside from supportive therapy to address the apparent symptoms, currently no active treatment exists for treating acute pancreatitis.

Attenuating the inflammatory response would be an important step towards reducing the extent of damage that results from pancreatic inflammation. It would, thus, be highly desirable to have effective methods available to attenuate the pancreatic inflammation.

### SUMMARY OF THE INVENTION

In one aspect of the present invention, provided herein are methods for treating an acute pancreatic inflammation in a subject, comprising the administration of a composition comprising a regulator of pancreatic interleukin-22 (IL-22) expression in a dosage and dosing regimen effective to attenuate pancreatic inflammation and pancreatic tissue damage. In one embodiment, the regulator of pancreatic IL-22 expression is an arylhydrocarbon (AhR) agonist such as biliverdin.

In a further aspect of the present invention, provided herein are methods for attenuating pancreatic inflammation in a subject at risk of developing a chronic pancreatic disorder, comprising the administration of a composition comprising a regulator of pancreatic interleukin-22 (IL-22) expression in a dosage and dosing regimen effective to attenuate pancreatic inflammation and to attenuate pancreatic tissue damage. In one embodiment, the regulator of pancreatic IL-22 expression is an aryl hydrocarbon (AhR) agonist such as biliverdin.

In another aspect of the present invention, provided herein are methods for attenuating or preventing acute lung injury that is associated with pancreatic inflammation in a subject, comprising the administration of a composition comprising a regulator of pancreatic interleukin-22 (IL-22) expression in a dosage and dosing regimen effective to attenuate said acute lung injury. In one embodiment, the regulator of pancreatic IL-22 expression is an aryl hydrocarbon (AhR) agonist such as biliverdin.

In certain aspects, the regulator of pancreatic IL-22 expression is a recombinant, isolated protein or an isolated biologically active fragment thereof; a recombinant, isolated peptide or an isolated biologically active fragment thereof, a peptidomimetic, or a small molecule.

The above summary is not intended to include all features and aspects of the present invention nor does it imply that the invention must include all features and aspects discussed in this summary.

### INCORPORATION BY REFERENCE

All publications mentioned in this specification are herein incorporated by reference to the same extent as if each individual publication was specifically and individually indicated to be incorporated by reference.

### BRIEF DESCRIPTION OF THE DRAWINGS

The accompanying drawings illustrate embodiments of the invention and, together with the description, serve to explain the invention. These drawings are offered by way of illustration and not by way of limitation; it is emphasized that the various features of the drawings may not be to-scale.

FIG. 1 illustrates, as detailed in Example 1, that IL-22 induces phosphorylation of signal transducer and activator of transcription 3 (STAT3) and RegIII (also known as pancreatitis-associated protein, PAP) genes in the pancreas. (A) Tissue lysates, obtained from Balb/c mice, were prepared from lung, liver, spleen, pancreas and colon and examined for the expression of IL-22RA1 by western blot, using α-tubulin as loading control. As demonstrated in panel A, the highest levels of expression of IL-22RA1 were observed in the pancreas with detectable expression in lung, liver, spleen and colon as well. (B) Balb/c mice were treated with recombinant IL-22 (rIL-22); 1 hour and 3 hours later, tissue extracts from pancreas, liver and spleen were prepared and levels of phosphorylation of STAT3 (p-STAT3) as well as total STAT3 levels in these tissues were examined by western blot. Significant STAT3 activation was observed in the pancreas at 1 hour following treatment with rIL-22. (C) Pancreas sections, removed from mice at 0 and 1 hour following treatment with rIL-22, as described in (B), were co-stained with phospho-STAT3 (pSTAT3; green) and DAPI (nuclei; blue) and assessed by confocal microscopy. The STAT3 activation which was observed in pancreas at 1 hour following treatment with rIL-22, as described in (B), was confirmed by this immunofluorescence staining method. (D) Primary pancreatic acinar cells, that were harvested from Balb/c mice, were incubated with 10 ng/ml rIL-22 for the indicated times of 0, 5, 15 and 30 minutes, and then lysed for immunoblotting with pSTAT3 and total STAT3. As can be seen here, pSTAT3 expression was induced by the rIL-22 treatment in pancreatic acinar cells in a time-dependent manner. (E) Primary pancreatic acinar cells were treated with different doses of rIL-22, ranging from 0-100 ng/ml, as indicated, for 15 min and then assayed for pSTAT3 and total STAT3 by western blotting. As can be seen here, pSTAT3 expression was induced by the rIL-22 treatment in pancreatic acinar cells in a dose-dependent manner. (F) Balb/c mice were treated intra-peritoneally (200 ng/mouse, administered in a concentration of 1 ng/μl) with PBS or rIL-22; 24 h following treatment the pancreas RNA was isolated and cDNA was prepared for quantitative PCR analysis. Results are shown as fold change in RegIIβ (PAP1) and RegIIIγ (PAP3) mRNA expression relative to the control group. As shown, rIL-22 treatment stimulated the expression of RegIIIβ (PAP1) and RegIIIγ (PAP3).

FIG. 2, as further described in Example 1, illustrates that recombinant IL-22 treatment did not stimulate the expression of serum amyloid A protein 3 (SAA3, FIG. 2A), β-defensin2 (FIG. 2B) and IL-22RA1 (FIG. 2C). Results are shown as fold change in SAA3, b-defensin2, and IL-22RA1 mRNA expression relative to the control group. Ns=nonsignificant. Mice were given PBS or rIL-22 (200 ng/mouse) intra-peritoneally. After 24 h, pancreas RNA was isolated and cDNA prepared for quantitative PCR analysis.

FIG. 3 illustrates, as further described in Examples 2 and 3, that pancreatic IL-22RA1 is upregulated, whereas IL-22 is downregulated during acute pancreatitis. (Panels A, B) Total pancreatic tissue homogenates were obtained from mice with induced pancreatitis, namely from pancreata of choline-deficient/DL-ethionine (CDE diet)-fed (A) and caerulein-treated (B) mice at the indicated times. Representative data from two age- and sex-matched mice are shown for each time point illustrating the expression of IL-22RA1 by western blot, using α-tubulin as loading control. The CDE diet was fed once to the mice just at the outset of the study (at 0 hours), while caerulein was administered intraperitoneally in hourly intervals, starting at 0 hours, then 1, 2, 3, 4, 5 hours with a last injection at 6 hours after the start of the study. (Panels C, D) Total pancreatic tissue homogenates were obtained from CDE-diet-fed (C) and caerulein-treated (D) mice at the indicated times. IL-22 expression levels were determined by ELISA. (E) Pancreatic leukocytes were isolated for IL-22 intracellular staining from Balb/c mice prior to and 24 h after CDE-diet feeding (3 mice were pooled per test/stain). Flow cytometry plots are those derived from sequential gating on total leukocytes (CD45.2+) and live cells as shown in FIG. 4. Iso indicates isotype control antibody staining (F) In the left graph, the quantity of IL-22+ cells is displayed as percent of total pancreatic leukocytes (CD45+ cells) that were isolated from mice fed with the CDE diet at times 0 and 24 h. In the right graph, absolute numbers of IL-22+ cells in pancreatic tissue are shown; data are derived from 3 pooled pancreata (3 mice). Shown are mean data±SEM from four independent experiments.

FIG. 4, panel A, shows the gating strategy used in FIG. 3E, as further described in Example 3. The gated CD45+IL-22+ population among the pancreatic leukocytes was further characterized by expression of CD4, CD11b, (CD3/CD19/CD4−)CD90/Sca-1 (to identify CD4⁻ LTi), and NKp46, as shown in panel B. In panel C, the frequency of IL-22+ cells among different leukocyte populations is shown.

FIG. 5 illustrates, as further described in Example 4, that the administration of exogenous, recombinant IL-22 attenuates established acute pancreatitis. (A) Balb/c mice were intraperitoneally injected with recombinant IL-22 (single dose, 200 ng/mouse, administered in a concentration of 1 ng/μl) or vehicle (PBS) at 24 h after feeding the CDE diet, and sacrificed at 60 h after feeding of the CDE diet. The bar graph shows results from serum lipase measurements. (B, C) Representative H&E staining of pancreas (B) and lung (C) tissue sections are shown. Scale bar: 100 μm. (D) Pancreatic tissue that was harvested from PBS- and rIL-22-treated mice at 60 h was processed for antibody staining of apoptotic cells (red, TUNEL assay) and nuclei (blue, DAPI). (E) The bar graph represents results of serum lipase measurements from isotype control (Iso) and anti-IL-22 mAb treated CDE mice. (F) Representative H&E stained pancreatic sections of isotype control (Iso) and anti-IL-22 mAb treated CDE mice are shown. Scale bar: 100 μm. All data is presented as mean±SEM of at least three independent experiments (n>5 mice per group and per experiment).

FIG. 6 illustrates, as further described in Example 4, the protective effect of pancreatic IL-22 in vivo in mice inflicted with acute pancreatitis. (A) Balb/c mice were treated with rIL-22 or PBS at 24 h after feeding CDE diet and sacrificed at 72 h. Percent survival is shown for the two groups. (B) Balb/c mice were treated with rIL-22/PBS at 24 h after CDE feeding and sacrificed at 60 h. Pancreas histology scores and (C) Lung myeloperoxidase (MPO) activity results are shown as bar graphs. (D) Bar graph represents pancreas IL-22 protein levels at 60 h following CDE feeding. (E,F) Balb/c mice were treated with anti-IL-22 mAb or isotype control (Iso) at 12 and 36 h after feeding CDE diet, and sacrificed at 60 h. Percent Survival (E) and pancreas histology scores (F) are shown. All data is presented as mean±SEM of at least three independent experiments (n>5 mice per group and per experiment).

FIG. 7 illustrates, as further described in Example 4, the protective effect of IL-22 in vitro on pancreatic acinar cells by delaying spontaneous apoptosis. Primary pancreatic acinar cells isolated from naive mice were treated with vehicle PBS (−) or 100 ng/mL-22 (+) for 2 h or 4 h in vitro. The cells were then lysed, proteins were separated by runing in SDS/PAGE and then transferred into a membrane for western blotting. As shown, Caspase-3 (cCasp3), an effector caspase that indicates actively occurring apoptosis, and α-tubulin, as loading control, were detected using specified antibodies.

FIG. 8 illustrates, as further described in Example 5, that administration of aryl hydrocarbon receptor (AhR) antagonist CH-223191 decreases pancreatic IL-22 production and worsens acute pancreatitis. (A) Balb/c mice were treated with vehicle control (VE) or AhR antagonist CH-223191 (100 μg/mouse, administered in a concentration of 0.5 μg/μl) on day 1 and 2 (once per day); pancreata were then harvested at day 3. Pancreatic leukocytes were isolated and gated on total leukocytes (CD45.2+) and then analyzed for the frequency of IL-22+ cells. Iso, intracellular staining with isotype control ab. (B) The bar graph represents IL-22+ cells as percent of total leukocytes from vehicle or CH-223191 treated mice. Data shown are mean±SEM from three or more independent experiments. (C) Balb/c mice were injected with vehicle control (VE) or AhR antagonist CH-223191 daily (arrows) prior to initiation of CDE-diet feeding. (D) After two days of CDE feeding, sera were collected for lipase measurement and presented as a bar graph. (E, F) Pancreata were also collected for H&E staining and histologic score evaluation. Scale bar: 100 μm. Data is presented as mean±SEM (n≧5 mice per group).

FIG. 9 as further described in Example 5, compares pancreatic IL-22 production in AhR non-responsive mice versus wildtype mice in the caerulein acute pancreatitis mouse model. (A) Pancreata from C57/B6 wild-type (WT) and AhR^{d} were collected for IL-22 determination by ELISA assay. (B) C57BL/6 wild-type (WT) and AhR^{d} mice were treated with caerulein (Cae) to induce acute pancreatitis and pancreatic IL-22 was determined by ELISA assay. (C) Balb/c mice were treated with vehicle control (VE) or AhR antagonist CH-223191 on day 1 and 2 (once per day, 100 μg/mouse). Pancreata were then harvested at day 3 and used for IL-22 determination by ELISA assay. (D) Balb/c mice were treated with vehicle control (VE) or AhR antagonist CH-223191 daily for two days prior to initiation of CDE feeding. After 2 days of CDE feeding, pancreata were isolated for determination of IL-22 by ELISA. Data is presented as mean±SEM or at least 3 independent experiments.

FIG. 10 illustrates, as further described in Example 5, that defective aryl hydrocarbon receptor (AhR) signaling accelerates acute pancreatitis. (A) Pancreatic leukocytes were isolated from C57BL/6 wild-type (WT) and AhR^{d} (aryl hydrocarbon receptor deficient) mice. Isolated cells were gated on live cells, leukocytes (CD45.2+) and then analyzed for frequency of IL-22+ cells. (B) The bar graph represents IL-22+ cells as a percent of total cells. (C, D) WT and AhR^{d} mice were injected with caerulein (1 μg/mouse/hour, 7 consecutive hourly injections) to induce acute pancreatitis. Sera and pancreata were collected for lipase measurement and H&E staining, respectively. Data is presented as mean±SEM from at least three independent experiments.

FIG. 11 illustrates, as further described in Example 5, illustrates reduced pancreatic IL-22 levels in aryl hydrocarbon receptor deficient mice. Pancreatic leukocytes were isolated from C57BL/6 wild-type (WT) and AhR^{-/-} mice and analyzed for frequency of IL22+ cells by flow cytometry. Isolated cells were gated on live cells, leukocytes (CD45.2+) and then analyzed for frequency of IL-22+ cells.

FIG. 12 illustrates, as further described in Example 6, that aryl hydrocarbon receptor (AhR) activation with AhR agonist biliverdin increases pancreatic IL-22 production and protects against acute pancreatitis. (A) Balb/c mice were treated with vehicle (VE) or AhR agonist biliverdin (BV) on day 1 and 2 (once per day). Pancreata were harvested at day 3 and used for IL-22 determination by ELISA (A) and by flow cytometry (B). (C) The bar graph represents IL-22+ cells as percent of total leukocytes from vehicle or BV treated mice. Data shown are mean±SEM from three or more independent experiments. (D) Balb/c mice were injected with vehicle control VE or BV 24 h after CDE-diet feeding and sacrificed at 60 h after feeding. (E) Serum lipase measurement is presented in vehicle versus BV-treated mice. (F) A representative H&E staining of pancreata from vehicle versus BV-treated mice is shown. Scale bar: 100 μm. Data is represented as mean±SEM (n=5 mice per group).

FIG. 13 illustrates, as further described in Example 6, that the aryl hydrocarbon receptor (AhR) mediates the protective function of pancreatic IL-22 in acute pancreatitis. (A) Balb/c mice were treated with biliverdin (BV) at time 0 and treated with isotype control (Iso) or anti-IL22 antibody at 12 and 36 h after CDE-diet feeding. Serum lipase measurement results are shown as a bar graph. (B) A representative of pancreata H&E staining is shown. Scale bar: 100 μm. (C) C57BL/6 wild-type (WT) and AhR^{d} mice underwent caerulein-induced AP. PBS or rIL-22 (single dose, 200 ng/mouse, administered in a concentration of 1 ng/μl) was administrated to WT or AhR^{d} mice, respectively, at the indicated times (arrows). Serum lipase measurement results are shown as a bar graph. (D) Representative H&E staining of the pancreata are shown, Scale bar: 100 μm. Data is presented as mean±SEM from three independent experiments with minimum of 5 mice per group. (E) Schematic representation summarizing findings of AhR activity and IL-22 cross talk in maintaining tissue repair and homeostasis in the pancreas.

### DETAILED DESCRIPTION

Before describing detailed embodiments of the invention, it will be useful to set forth definitions that are utilized in describing the present invention.

### DEFINITIONS

The practice of the present invention may employ conventional techniques of chemistry, molecular biology, recombinant DNA, microbiology, cell biology, immunology and biochemistry, which are within the capabilities of a person of ordinary skill in the art. Such techniques are fully explained in the literature. For definitions, terms of art and standard methods known in the art, see, for example, Sambrook and Russell ‘Molecular Cloning: A Laboratory Manual’, Cold Spring Harbor Laboratory Press (2001); ‘Current Protocols in Molecular Biology’, John Wiley & Sons (2007); William Paul ‘Fundamental Immunology’, Lippincott Williams & Wilkins (1999); M. J. Gait ‘Oligonucleotide Synthesis: A Practical Approach’, Oxford University Press (1984); R. Ian Freshney “Culture of Animal Cells: A Manual of Basic Technique’, Wiley-Liss (2000); ‘Current Protocols in Microbiology’, John Wiley & Sons (2007); ‘Current Protocols in Cell Biology’, John Wiley & Sons (2007); Wilson & Walker ‘Principles and Techniques of Practical Biochemistry’, Cambridge University Press (2000); Roe, Crabtree, & Kahn ‘DNA Isolation and Sequencing: Essential Techniques’, John Wiley & Sons (1996); D. Lilley & Dahlberg ‘Methods of Enzymology: DNA Structure Part A: Synthesis and Physical Analysis of DNA Methods in Enzymology’, Academic Press (1992); Harlow & Lane ‘Using Antibodies: A Laboratory Manual: Portable Protocol No. I’, Cold Spring Harbor Laboratory Press (1999); Harlow & Lane ‘Antibodies: A Laboratory Manual’, Cold Spring Harbor Laboratory Press (1988); Roskams & Rodgers ‘Lab Ref: A Handbook of Recipes, Reagents, and Other Reference Tools for Use at the Bench’, Cold Spring Harbor Laboratory Press (2002). Each of these general texts is herein incorporated by reference.

Unless defined otherwise, all technical and scientific terms used herein have the same meaning as commonly understood by a person of ordinary skill in the art to which this invention belongs. The following definitions are intended to also include their various grammatical forms, where applicable. As used herein, the singular forms “a” and “the” include plural referents, unless the context clearly dictates otherwise.

The term “regulator of pancreatic Interleukin-22 production”, as used herein, relates to a molecule that is capable of regulating Interleukin-22 production in the pancreatic tissue, particularly, of increasing Interleukin-22 production in the pancreatic tissue.

The term “to attenuate”, as used herein, refers to its general dictionary meaning of “to reduce or to decrease in force, amount, degree.”

The term “aryl hydrocarbon receptor agonist”, as used herein, relates to biologically active, recombinant, isolated peptides and proteins, including their biologically active fragments, peptidomimetics and small molecules that are capable of stimulating the aryl hydrocarbon receptor and, thereby, cause aryl hydrocarbon receptor activation.

The term “pharmaceutical composition”, as used herein, refers to a mixture of a regulator of pancreatic Interleukin-22 production with chemical components such as diluents or carriers that do not cause unacceptable, i.e. counterproductive to the desired therapeutic effect, adverse side effects and that do not prevent the aryl hydrocarbon receptor agonist from exerting a therapeutic effect. A pharmaceutical composition serves to facilitate the administration of the regulator of pancreatic Interleukin-22 production, which in some embodiments is an arylhydrocarbon receptor agonist.

The term “therapeutic effect”, as used herein, refers to a consequence of treatment that might intend either to bring remedy to an injury that already occurred or to prevent an injury before it occurs. A therapeutic effect may include, directly or indirectly, the reduction of pancreatic inflammation (pancreatitis) and reduction of damage of pancreatic tissue following acute or chronic injury. A therapeutic effect may also include, directly or indirectly, the arrest, reduction, or elimination of the progression of pancreatic cell death following acute or chronic injury

The terms “therapeutically effective amount” and “dosage effective to attenuate pancreatic inflammation” relate to an amount of a regulator of pancreatic Interleukin-22 production that is sufficient to provide a desired therapeutic effect in a subject. Naturally, dosage levels of the particular regulator of pancreatic Interleukin-22 production employed to provide a therapeutically effective amount vary in dependence of the type of injury, the age, the weight, the gender, the medical condition of the human subject, the severity of the condition, the route of administration, and the particular regulator of pancreatic Interleukin-22 production employed. Therapeutically effective amounts of a regulator of pancreatic Interleukin-22 production, as described herein, can be estimated initially from cell culture and animal models. For example, IC₅₀ values determined in cell culture methods can serve as a starting point in animal models, while IC₅₀ values determined in animal models can be used to find a therapeutically effective dose in humans.

The term “dosing regimen”, as used herein, refers to the administration schedule and administration intervals of the particular regulator of pancreatic Interleukin-22 production employed to obtain the desired therapeutic effect.

The term “analog of biliverdin” or “analog of bilirubin” refers to molecules that are similar in chemical structure (“structural analog”) to biliverdin or bilirubin, which are endogenous bile pigments.

The term “recombinant”, as used herein, relates to a protein or polypeptide that is obtained by expression of a recombinant polynucleotide.

The terms “isolated” and “purified”, as used herein, relate to molecules that have been manipulated to exist in a higher concentration or purer form than naturally occurring.

Subject at risk of developing a chronic pancreatic disorder are defined as individuals who have experienced at least one case of acute pancreatitis.

The term “attenuating” as used herein, is employed in the meaning of decreasing, alleviating, relieving, protecting from.

### Interleukin-22 (IL-22)

IL-22 is a member of the IL-10 cytokine family and plays a critical role in modulating the tissue response during inflammation. IL-22 is an important cytokine allowing for cross talk between leukocytes and epithelial cells, since IL-22 production and receptor expression are restricted to leukocytes and epithelial cells, respectively (Liang et al., 2006). In addition to the well studied Th17 cells, various other leukocyte subsets such as γδ T cells, Th22 cells, NK cells, monocytes, dendritic cells and lymphoid tissue-inducer (LTi) cells have been shown to be an important source of IL-22 (Zenewicz et al., 2008).

The bioavailability of IL-22 is regulated by a soluble IL-22 binding protein (IL-22BP) that acts as a natural antagonist (Xu et al., 2001). When IL-22 is secreted together with pro-inflammatory agents such as TNF-α, IFN-γ and/or IL-17, it has been observed to contribute to a dramatic increase in the inflammatory immune reaction. In contrast, when IL-22 alone is secreted alone, it rather has protective and regenerative effects particularly on epithelial cells (Eyerich et al., 2009; Nograles et al., 2008).

### Assessing the Extent of IL-22 Production

IL-22 exerts its actions upon binding to a receptor complex composed of a type-1 receptor chain (IL-10Rβ) and a type-2 receptor chain (IL-22RA1) (Xie et al., 2000). Upon binding to its receptor complex, IL-22 induces phosphorylation of tyrosine kinases Jak1 and Tyk2 (LeJeune et al., 2002), which results in activation of signal transducer and activator of transcription (STAT)3 and, depending on the system, STAT1 or STAT5 (Dumoutier et al., 2000; Zheng et al., 2007). IL-22 also induces the three major MAP kinase pathways (Mek/Erk, JNK, p38 kinase) (LeJeune et al., 2002).

IL-22 receptor activation leads to STAT3 mediated proliferative and anti-apoptotic pathway signaling as well as anti-microbial induction that help prevent damage and aid tissue repair (LeJeune et al., 2002). While IL-10R2 is expressed ubiquitously in various organs, IL-22RA1 has a more restricted expression with highest level of mRNA reported in the pancreas followed by the intestines and the skin (Gurney A, 2004). Despite these findings and detailed studies for example outlining importance of IL-22 in gut immunity (Sonnenberg et al., 2011), regulation of IL-22 and activation of IL-22 receptor in the pancreas under both homeostatic and inflammatory states has not been elucidated.

### Pancreatic IL-22 Protects Against Acute Pancreatitis and Pancreatitis-Associated Lung Injury

As described herein in detail in Examples 4 and 6, IL-22 was found to exert a protective role against pancreatic inflammation in the pancreas and to attenuate acute pancreatitis as well as pancreas-associated lung injury in mice. The regenerative and healing effects of IL-22 in lung injury are thought to be achieved by increasing transepithelial resistance to injury and by promoting barrier function through the induction of epithelial cell proliferation (Aujla et al., 2008). It was, furthermore, found that activation of the aryl hydrocarbon receptor was instrumental in the pancreatic Il-22 production and required for IL-22's protective function in the pancreas.

IL-22's protective and regenerative effects comprise inducing antimicrobial peptides, inducing re-epithelialization and enhancing the migration and proliferation of epithelial cells.

### The Pancreas: A Dual-Functioning Gland

The pancreas is both an exocrine and an endocrine gland organ in the digestive system and endocrine system, exhibiting two different types of pancreatic tissue.

Functions of the Pancreas as an Exocrine Gland.

As an exocrine gland, consisting of miniscule ducts, the so-called pancreatic acini, that are surrounded by the pancreatic acinar cells, the pancreas produces, stores and secretes digestive enzymes into the duodenum to assist in the breakdown of food and absorption of nutrients. The pancreatic acinar cells are small and dark-staining cells that form berry-like clusters. The pancreatic acinar cells, which secrete proteolytic enzymes, lipases and α-amylases for the hydrolysis of food constitutents into proteins, fat and carbohydrates, comprise about 80% of the pancreas. Pancreatic acinar cells express IL-22RA1 mRNA and have been shown to be a target for IL-22 action in vitro (Aggarwal et al., 2001). Pancreatic stellate cells reside in exocrine areas of the pancreas and are myofiberblast-like cells that can switch between a quiescent and an activated phenotype. Pancreatic stellate cells can be activated through paracrine factors, such as cytokines (IL-1, IL-6, IL-8, and TNF-α), growth factors (PDGF and TGF-β₁), angiotensin II, and reactive oxygen species that were released by damaged neighboring cells and leukocytes recruited in response to pancreatic injury. Activated pancreatic stellate cells proliferate and migrate to areas of injury within the pancreas, where they participate in tissue repair activities such as by secreting extracellular matrix components to promote tissue repair. Pancreatic stellate cells may play a role in the pathogenesis of pancreatitis and pancreatic cancer (Omary et al., 2007).

Functions of the Pancreas as an Endocrine Gland.

As an endocrine gland, consisting of various cell type clusters called islets of Langerhans, that encompass α-cells, β-cells, γ-cells and δ-cells, the pancreas produces and secretes various hormones: α-cells secrete glucagon to increase glucose levels in the blood, while β-cells secrete insulin to decrease glucose blood levels. γ-cells secrete pancreatic polypeptide in order to regulate pancreatic endocrine and exocrine secretion activities and δ-cells secrete somatostatin in order to regulate the activity of the α-cells and β-cells.

### Pancreatic Inflammation Disorders and Pancreatitis-Associated Disorders

Pancreatic inflammation manifests itself in an acute form, as acute pancreatitis, and in a chronic form, as chronic pancreatitis.

### Acute Pancreatitis

Disturbances of the pancreatic acinar cell function become primarily manifest as acute pancreatitis. Insults due to chemical exposure, auto-immune reactions and surgical procedures that are assumed to primarily harm the pancreatic acinar cells have been found to result in acute pancreatitis (Leung & Chan, 2009).

Acute pancreatitis is a sudden inflammation of the pancreas, whose clinical course can vary from mild symptoms to a complete organ failure with potential fatal consequences (Papachristou, 2008). Other clinical disorders that can result from pancreatic acinar cell dysfunction include chronic pancreatitis, autoimmune pancreatitis, pancreatic exocrine insufficiency and pancreatic cancers.

Acute pancreatitis is an inflammatory disease of the pancreatic acinar cells that is characterized by fluid accumulation, hemorrhage and cell necrosis in the pancreatic tissue. Besides drug-induced acute pancreatitis, gallstones and heavy alcohol consumption are among the leading causative factors.

Under regular, physiological conditions, pancreatic proteolytic enzymes are secreted as inactive precursors into the duodenum where enterokinase, an enzyme located along the brush border of duodenal enterocytes, initiates their activation (Leung & Ip, 2006). While the development of acute pancreatitis is still not fully resolved, it seems plausible that proteolytic enzymes might become prematurely activated while they are still within the pancreas, thus leading to a gradual autodigestion of the gland. Acute pancreatitis manifests itself in various forms. Edematous pancreatitis which is characterized by interstitial edema is a mild form, where the structure of the pancreatic acinar cells mostly stays intact.

In the case of severe acute pancreatits, the pancreatic acinar cells are severely damaged which leads to a release of inflammatory mediators such as reactive oxygen species, an infiltration of leukocytes and a release of proinflammatory cytokines such as tumor necrosis factor-alpha (TNF-α), which is expressed in pancreatic acinar cells. Other pro-inflammatory cytokines that have been found to play a critical role in the pathogenesis of acute pancreatitis by driving the subsequent inflammatory response are Interleukin-1 (IL-1), IL-6, IL-8, IL-10 and monocyte chemotactic protein-1 (MCP-1) (Papachristou, 2008).

Current treatment options of acute pancreatitis consist primarily of symptomatic therapies. Since the insulin secretion by the β-cells might be impaired, transient or chronic insulin replacement therapy is usually indicated.

### Chronic Pancreatitis

Chronic pancreatitis is assumed to primarily result from repeated episodes of acute pancreatic inflammation, where acute phases of pancreatic inflammation, that are accompanied by pancreatic necrosis due to acute pancreatitis, are followed by the development of pancreatic fibrosis. Chronic pancreatitis leads over time to a significant alteration of the pancreas' normal structure and function, and, consequently, to an impairment of both the exocrine and endocrine functions of the pancreas (Witt et al., 2007; Mews, 2002). An overactivation of pancreatic stellate cells is also discussed as a contributory factor to chronic pancreatitis (Leung & Chan, 2009).

Subjects suffering from chronic pancreatitis are generally characterized by malabsorption and corresponding weight loss and might experience abdominal pain which might be transient or constant.

The treatment of chronic pancreatitis is geared towards relieving the abdominal pain with analgesics and alleviating malabsorption by supplementation with pancreatic enzymes; surgical removal of fibrotic pancreatic tissue possibly followed by transplantation of the subject's own insulin-producing beta cells surgery present further treatment options. Since the insulin secretion by the β-cells might be severely impaired, chronic insulin replacement therapy might be indicated as well.

Autoimmune pancreatitis is considered a subtype of chronic pancreatitis that responds to treatment with corticosteroids. Clinically, increased levels of immunoglobulins and serum autoantibodies have been identified.

### Exocrine Pancreatic Insufficiency

Exocrine pancreatic insufficiency, which eventually results from chronic pancreatitis and its progressive loss of digestive enzyme-producing pancreatic cells, is the inability to properly digest food due to a lack of digestive enzymes from the pancreas and is found with particular frequency in individuals suffering from cystic fibrosis. Loss of digestive enzymes leads to maldigestion and malabsorption of nutrients.

### Pancreatic Cancer

Ductual adenocarcinoma is the most common form of pancreatic cancer and is assumed to arise from progressive tissue changes as they occur in both acute and chronic pancreatitis, rendering the identification of effective treatment options for acute pancreatitis and possibly also for chronic pancreatitis highly important.

### Pancreatitis-Associated Acute Lung Injury and Acute Respiratory Distress Syndrome

Severe acute pancreatitis is often associated with an acute lung injury that can clinically become manifest as the acute respiratory distress syndrome with symptoms such as severe breathing difficulties and hypoxemia. Through the release of inflammatory mediators such as reactive oxygen species, an infiltration of leukocytes and a release of proinflammatory cytokines, as already described supra under ‘acute pancreatitis’, the inflammation assumes a systemic nature allowing the pancreatic injury to extend to distant organs such as the lung, often causing multiple organ failure.

Acute lung injury is generally characterized by a disruption of the alveolar-capillary interface, leakage of protein-rich fluid into the interstitium and alveolar space, and extensive release of cytokines and migration of neutrophils.

### Aryl Hydrocarbon Receptor (AhR)

### AhR's Toxicological Role

The aryl hydrocarbon receptor (Ahr) is a cytosolic ligand-activated transcription factor that recognizes various small low-molecular weight synthetic compounds as well as natural molecules as ligands and activators of AhR signaling. In the absence of a ligand, the AhR is contained in the heteromeric unliganded aryl hydrocarbon receptor complex (AHRC) (Hankinson, 1995). Ligand binding, such as binding of polycyclic or halogenated aromatic hydrocarbons, causes a conformational change and release of AhR from the complex, upon which AhR translocates to the nucleus and binds to its dimerization partner ARNT, also known as HIF-1beta. Ahr/ARNT heterodimers then bind within regulatory domains of genes coding for phase-1- and phase-II-metabolizing enzymes and modify gene expression (Hankinson, 1995). Most typically, phase-I metabolizing enzymes such as the P450 enzymes are upregulated by ligands for AhR, while phase-II-metabolizing enzymes, whose task it is to render molecules more hydrophilic for excretion out of the body as to detoxify the body, are downregulated. As a consequence, the metabolism and excretion of harmful molecules is substantially delayed and their residence time in the body substantially elevated, increasing the chance of harmful effects for the body.

The aryl hydrocarbon receptor is well known for mediating the herbicide 2,3,7,8-tetrachlorodibenzo-p-dioxin (TCDD)'s toxicity or, in short, dioxin's toxicity as well as the toxicological effects of many other halogenated, organic, aromatic or antiaromatic hydrocarbons, usually environmental pollutants of some sort, which can lead to carcinogenic DNA mutations with subsequent activation of proto-oncogenes or by inactivation of tumor suppressor genes (Hankinson, 1995). Other harmful effects can be suppression of the immune system, particularly the cell-based immune system, teratogenesis and modulation of hormonal effects.

### AhR's Physiological Role

It is important to note that many regular dietary components such as flavonoids and indoles, from fruits and vegetables, are AhR ligands as well, and so are endogenous compounds such as biliverdin and bilirubin. These findings gave rise to the discovery that the aryl hydrocarbon receptor has a physiological role in the normal development of mammalian as well (Nguyen & Bradfield, 2008).

In elegant studies by several research groups, AhR has been established as a critical ligand dependent transcription factor for IL-22 production (Monteleone et al., 2011; Alam et al., 2010). As described herein in detail in Examples 5 and 6, supra, the aryl hydrocarbon receptor was found to considerably increase pancreatic IL-22 production and to mediate IL-22's protective function during pancreatic inflammation. Consistent with these results of IL-22's protective role in ongoing severe acute pancreatitis and pancreatic acinar cell apoptosis, AhR antagonism with either chemical blockade or use of AhR non-responsive transgenic mice led to a significant decrease in pancreatic IL-22 production and worsening of pancreatitis. Furthermore, biliverdin, an endogenous AhR ligand, was shown to increase pancreatic IL-22 production and to attenuate acute pancreatitis. Furthermore, AhR activation was shown and proven to increase IL-22 production in the pancreas and to mediate the protective role of IL-22 in acute pancreatitis using two independent approaches by reconstituting AhR non-responsive mice with IL-22 and neutralizing IL-22 in mice where AhR had been activated.

### Aryl Hydrocarbon Receptor Agonists (AhR Agonists)

The present invention provides methods for attenuating pancreatic inflammation and pancreatic damage using regulators of pancreatic Interleukin-22 production. In some embodiments, aryl hydrocarbon receptor agonists (AhR agonists) are such regulators of pancreatic IL-22 production.

The inventors of the present invention have found that the activation of the aryl hydrocarbon receptor leads to an increase in the IL-22 production in the pancreas. As described in the below following Examples, the administration of an AhR agonist, such as the endogenously occurring biliverdin, was determined to significantly attenuate pancreatic inflammation in mice. The results indicate that a composition comprising an agent that increases pancreatic interleukin-22 production in the pancreas, such as an agonist of the aryl hydrocarbon receptor, is useful to attenuate inflammation in the pancreatic tissue and to prevent damage to the pancreatic tissue such as necrosis and apoptosis of pancreatic acini cells. The results also indicate that pancreatic inflammation may be prevented altogether by means of administering regulators of Interleukin-22 production such as AhR agonists.

Aryl hydrocarbon receptor agonists may be biologically active, recombinant, isolated peptides and proteins, including their biologically active fragments, peptidomimetics or small molecules. In the working examples, the endogenously occurring biliverdin was utilized as an AhR agonist to activate the aryl hydrocarbon receptor.

AhR receptor agonists can be identified experimentally using a variety of in vitro and/or in vivo models. Isolated AhR agonists can be screened for binding to various sites of the purified AhR proteins. Compounds that can be utilized in the context of the present invention to attenuate acute pancreatitis can also be functionally screened for their ability to exert anti-inflammatory effects through increased IL-22 production using pancreatic acini in vitro culture systems as well as in vivo animal models of acute pancreatitis (e.g., monkey, rat, or mouse models). Candidate compounds that exert such desired effects may also be identified by known pharmacology, structure analysis, or rational drug design using computer based modeling.

Candidate compounds that exert such anti-inflammatory and IL-22 production increasing effects may encompass numerous chemical classes, though typically they are organic molecules, preferably small organic compounds having a molecular weight of more than 50 and less than about 2,500 daltons. They may comprise functional groups necessary for structural interaction with proteins (e.g., hydrogen bonding), and typically include at least an amine, carbonyl, hydroxyl, or carboxyl group. They often comprise cyclical carbon or heterocyclic structures and/or aromatic or polyaromatic structures substituted with one or more functional groups. They may be found among biomolecules including peptides, saccharides, fatty acids, steroids, purines, and pyrimidines, and structural analogs thereof.

Candidate compounds that exert anti-inflammatory effects and IL-22 production increasing effects in the pancreas can also be synthesized or isolated from natural sources (e.g., bacterial, fungal, plant, or animal extracts). The synthesized or isolated candidate compound may be further chemically modified (e.g., acylated, alkylated, esterified, or amidified), or substituents may be added (e.g., aliphatic, alicyclic, aromatic, cyclic, substituted hydrocarbon, halo (especially chloro and fluoro), alkoxy, mercapto, alkylmercapto, nitro, nitroso, sulfoxy, sulfur, oxygen, nitrogen, pyridyl, furanyl, thiophenyl, or imidazolyl substituents) to produce structural analogs, or libraries of structural analogs (see, for example, U.S. Pat. Nos. 5,958,792; 5,807,683; 6,004,617; 6,077,954). Such modification can be random or based on rational design (see, for example, Cho et al., 1998; Sun et al., 1998).

Aryl hydrocarbon receptor agonists may be administered, for example locally into the pancreas or systemically, in a dosage and dosage regimen that is effective to provide the desired anti-inflammatory effects and IL-22 production increasing effects in the pancreas.

Known AhR agonists, also called AhR ligands, encompass naturally occurring as well as synthetic molecules. Examples of naturally occurring AhR agonists include biliverdin and bilirubin. Synthetic AhR agonists include members of polyhalogenated dibenzo-p-dioxins, dibenzofurans, biphenyls, benzo(a)pyrene, benzanthracenes, and benzoflavones such as the before mentioned herbicide 2,3,7,8-tetrachlorodibenzo-p-dioxin (TCDD), di-indolyl-methane (DIM) that exhibits anticancer properties, alkyl-polychlorodibenzofurans (alkyl-PCDFs) such as 6-Methyl-1,3,8-trichlorodibenzofuran (6-MCDF), 8-Methyl-1,3,8-trichlorodibenzofuran (8-MCDF) which have anti-estrogenic effects (Denison & Nagy, 2003).

### Assessing Pancreatic Inflammation

As discussed in Example 6, aryl hydrocarbon receptor activation had a distinct anti-inflammatory effect and significantly increased the production of protective IL-22 in the pancreas of mice. The degree of pancreatic inflammation and the degree of attenuating pancreatic inflammation following administration of aryl hydrocarbon receptor agonists can, for instance, be assessed by measuring levels of proinflammatory cytokines, such as IL-1α, IL-10, IL-6 and TNF-α, prior to the administration of an AhR agonist and at specified time(s) after the administration of an AhR agonist. Such specified time can be hours, days or weeks after the administration of an AhR agonist, and the AhR agonist can be administered once a day or multiple times per day.

Pancreatic inflammation can, for example, be assessed, as demonstrated in the various examples and figures herein, by measuring serum lipase levels. Elevated serum lipase levels indicate pancreatic inflammation and so do elevated serum amylase levels. Other indicators of pancreatic inflammation are increased recruitment of neutrophils to the pancreas, hypovolemia from capillary permeability, acute respiratory distress syndrome, disseminated intravascular coagulations, renal failure, cardiovascular failure, severe abdominal pain and gastrointestinal hemorrhage.

The attenuation of pancreatic inflammation can be determined by measuring the parameters mentioned above, used to assess pancreatic inflammation, such as serum lipase or serum amylase levels, before and after treatment with a regulator of pancreatic IL-22 production. Also, by comparing some of the above mentioned indicators of pancreatic inflammation, such as gastrointestinal hemorrhage, before and after treatment with a regulator of pancreatic IL-22 production. In all these scenarios, the treatment with a regulator of pancreatic IL-22 production might require repeated administration until a therapeutic effect is observed.

### Dosages, Dosing Regimens, Formulations and Administration of Regulators of IL-22 Expression, Including Aryl Hydrocarbon Receptor Agonists

The dosage and dosing regimen for the administration of a regulator of IL-22 expression for attenuating pancreatic inflammation, as provided herein, is selected by one of ordinary skill in the art, in view of a variety of factors including, but not limited to, age, weight, gender, and medical condition of the subject, the severity of the inflammatory response that is experienced, the route of administration (oral, systemic, local), the dosage form employed, and may be determined empirically using testing protocols, that are known in the art, or by extrapolation from in vivo or in vitro tests or diagnostic data.

The dosage and dosing regimen for the administration of a regulator of IL-22 expression, as provided herein, is also influenced by toxicity in relation to therapeutic efficacy. Toxicity and therapeutic efficacy can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Molecules that exhibit large therapeutic indices are generally preferred.

The therapeutically effective dose of a regulator of IL-22 expression can, for example, be less than 50 mg/kg of subject body mass, less than 40 mg/kg, less than 30 mg/kg, less than 20 mg/kg, less than 10 mg/kg, less than 5 mg/kg, less than 3 mg/kg, less than 1 mg/kg, less than 0.3 mg/kg, less than 0.1 mg/kg, less than 0.05 mg/kg, less than 0.025 mg/kg, or less than 0.01 mg/kg. Therapeutically effective doses of a regulator of IL-22 expression, administered to a subject as provided in the methods herein can, for example, can be between about 0.001 mg/kg to about 50 mg/kg. In certain embodiments, the therapeutically effective dose is in the range of, for example, 0.005 mg/kg to 10 mg/kg, from 0.01 mg/kg to 2 mg/kg, or from 0.05 mg/kg to 0.5 mg/kg. In various embodiments, an effective dose is less than 1 g, less than 500 mg, less than 250 mg, less than 100 mg, less than 50 mg, less than 25 mg, less than 10 mg, less than 5 mg, less than 1 mg, less than 0.5 mg, or less than 0.25 mg per dose, which dose may be administered once, twice, three times, or four or more times per day. In certain embodiments, an effective dose can be in the range of, for example, from 0.1 mg to 1.25 g, from 1 mg to 250 mg, or from 2.5 mg to 1000 mg per dose. The daily dose can be in the range of, for example, from 0.5 mg to 5 g, from 1 mg to 1 g, or from 3 mg to 300 mg.

In some embodiments, the dosing regimen is maintained for at least one day, at least two days, at least about one week, at least about two weeks, at least about three weeks, at least about one month, three months, six months, one year, three years, six years or longer. In some embodiments, an intermittent dosing regimen is used, i.e., once a month, once every other week, once every other day, once per week, twice per week, and the like.

Regulators of IL-22 expression or pharmaceutical compositions containing regulators of IL-22 expression may be administered to a subject using any convenient means capable of resulting in the desired attenuation of pancreatic inflammation as well as attenuation of pancreatic damage, also attenuation or prevention of acute lung injury. Routes of administration of a regulator of IL-22 expression or pharmaceutical compositions containing regulators of IL-22 expression include, but are not limited to, oral, nasal and topical administration and intramuscular, subcutaneous, intravenous, or intraperitoneal injections. A regulator of IL-22 expression or pharmaceutical compositions containing a regulator of IL-22 expression may also be administered locally at the site of inflammation.

The regulator of IL-22 expression may be administered in a single daily dose, or the total daily dose may be administered in divided doses, two, three, or more times per day. Optionally, in order to reach a steady-state concentration in the target tissue quickly, an intravenous bolus injection of the regulator of IL-22 expression can be administered followed by an intravenous infusion of the regulator of IL-22 expression.

The regulator of IL-22 expression can be administered to the subject as a pharmaceutical composition that includes a therapeutically effective amount of the regulator of IL-22 expression in a pharmaceutically acceptable vehicle. It can be incorporated into a variety of formulations for therapeutic administration by combination with appropriate pharmaceutically acceptable carriers or diluents, and may be formulated into preparations in solid, semi-solid, liquid, or gaseous forms, such as tablets, capsules, powders, granules, ointments, solutions, suppositories, injections, inhalants, gels, microspheres, and aerosols.

In some embodiments, a regulator of IL-22 expression can be formulated as a delayed release formulation. Suitable pharmaceutical excipients and unit dose architecture for delayed release formulations may include those described in U.S. Pat. Nos. 3,062,720 and 3,247,066. In other embodiments, the regulator of IL-22 expression can be formulated as a sustained release formulation. Suitable pharmaceutical excipients and unit dose architecture for sustained release formulations include those described in U.S. Pat. Nos. 3,062,720 and 3,247,066. The regulator of IL-22 expression can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat. No. 4,767,628), poly(ε-caprolactone) and poly(alkylene oxide) (U.S. 20030068384) to create a sustained release formulation. Such formulations can be used in implants that release an agent over a period of several hours, a day, a few days, a few weeks, or several months depending on the polymer, the particle size of the polymer, and the size of the implant (see, e.g., U.S. Pat. No. 6,620,422). Other sustained release formulations are described in EP 0 467 389 A2, WO 93/241150, U.S. Pat. No. 5,612,052, WO 97/40085, WO 03/075887, WO 01/01964A2, U.S. Pat. No. 5,922,356, WO 94/155587, WO 02/074247A2, WO 98/25642, U.S. Pat. Nos. 5,968,895, 6,180,608, U.S. 20030171296, U.S. 20020176841, U.S. Pat. Nos. 5,672,659, 5,893,985, 5,134,122, 5,192,741, 5,192,741, 4,668,506, 4,713,244, 5,445,832 4,931,279, 5,980,945, WO 02/058672, WO 9726015, WO 97/04744, and. US20020019446. In such sustained release formulations microparticles of drug are combined with microparticles of polymer. Additional sustained release formulations are described in WO 02/38129, EP 326 151, U.S. Pat. No. 5,236,704, WO 02/30398, WO 98/13029; U.S. 20030064105, U.S. 20030138488A1, U.S. 20030216307A1,U.S. Pat. No. 6,667,060, WO 01/49249, WO 01/49311, WO 01/49249, WO 01/49311, and U.S. Pat. No. 5,877,224.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients, and the like. The compositions can also include additional substances to approximate physiological conditions, such as pH adjusting and buffering agents, toxicity adjusting agents, wetting agents, and detergents. The composition can also include any of a variety of stabilizing agents, such as an antioxidant for example. Tablet formulations can comprise a sweetening agent, a flavoring agent, a coloring agent, a preservative, or some combination of these to provide a pharmaceutically elegant and palatable preparation.

Further guidance regarding formulations that are suitable for various types of administration can be found in Remington's Pharmaceutical Sciences, Mace Publishing Company, Philadelphia, Pa., 20th ed. (2000).

Formulations suitable for parenteral administration include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in-vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. In the following, experimental procedures and examples will be described to illustrate parts of the invention.

### EXPERIMENTAL PROCEDURES

The following methods and materials were used in the examples that are described further below.

Mice.

Mice including Balb/c, C57B6/J and AhR^{d} were purchased from Jackson laboratory and housed under pathogen-free conditions. All animal experiments were approved by Stanford University institutional animal care and use committees. Acute pancreatitis models. For caerulein-induced pancreatitis, age and sex matched mice were fasted for 12-16 hours. Mice then received 7 hourly intra-peritoneal injections of saline (control) or 50 μg/kg caerulein in saline and followed up to 12 h or indicated times. For the choline-deficient diet supplemented with DL-ethionine (CDE-diet) model of pancreatitis, young female mice (16-20 g) were fasted then fed a choline-deficient diet (Harlan Teklad) supplemented with 0.5% DL-ethionine (Sigma-Aldrich) or normal chow (control group) (Habtezion et al. (2011), Nakamichi et al., 2005).

Mice Treatments.

Mice were given PBS or recombinant IL-22 (200 ng/mouse, 1 ng/μl, Miltenyi Biotech) intra-peritoneally at 24 h after onset of CDE-diet-induced acute pancreatitis. For experiments involving anti-IL-22 mAb, mice were treated with 1 μg of either anti-IL-22 or isotype control mAb (R&D Systems) at indicated times. CH-223191 (100 μg/mouse, 0.5 μg/μl, Sigma-Aldrich) was dissolved in DMSO and Biliverdin hydrochloride (35 mg/kg, Frontier Scientific) in 20 mM NaOH adjusted to pH of 7.0. CH-223191, Biliverdin hydrochloride, or vehicle control were used to treat normal mice or mice undergoing AP at indicated times.

Histology and Immunofluorescence.

Mice were euthanized by CO₂ inhalation. Pancreata and other tissues were rapidly removed. Pancreas and lung pieces were immediately fixed in 10% formalin and embedded in paraffin. Fixed tissues were sectioned and stained with hematoxylin and eosin (H&E; performed by Histo-Tec Laboratory). The severity of pancreatitis and lung injury were scored blindly as described previously (Habtezion et al. (2011), Nakamichi et al., 2005). Other pancreas pieces were frozen in Tissue-Tek OCT compound and sectioned for fluorescence staining Immunofluorescence staining for p-STAT3 (Cell Signaling) and DAPI (nuclei) was performed according to manufacturer's guidelines. TUNEL assay was performed according to the manufacturer's instructions (ApopTag® Red In Situ Apoptosis Detection Kit, Millipore).

Biochemical Analysis and Myeloperoxidase (MPO) Activity Assay.

Blood was collected by intracardiac puncture, and serum was isolated for subsequent lipase level determination by diagnostic laboratory at Stanford University. Lung tissues were collected and MPO assay performed according to manufacture's guidelines (Biovision).

Western Blotting and ELISA.

Mouse pancreata were isolated and frozen immediately in liquid nitrogen. Total tissue and primary pancreatic acinar cells, isolated as described below, were homogenized in RIPA buffer containing protein inhibitors and analyzed by western blot as described previously (Nakamichi et al., 2005; Xue et al., 2010). IL-22RA1 and cleaved caspase-3 antibodies were purchased from Abcam and Cell Signaling, respectively. Supernatant from pancreas homogenate was analyzed using ELISA kit for mouse IL-22 (Biolegend).

Quantitative PCR.

Pancreatic tissue was lysed with Trizol reagent (Invitrogen) for total RNA preparation according to manufacturer's instructions. Briefly, cDNA was generated using GoScript reverse transcription system (Promega). Quantitative PCR was performed with an ABI-7900 Sequence Detection System (Applied Biosystems) using designed specific TaqMan probes and primers as follows: RegIIIβ (Forward, 5′-TGGAAGACAGACAAGATGCTG-3′; Reverse, 5′-TAAGAGCATCAGGCAGGAGA-3′; Probe, 5′-CCTCCAACAGCCTGCTCCGTC-3′); RegIIIγ (Forward, 5′-TCCTGTCCTCCATGATCAAA-3′; Reverse, 5′-TGGGTTCATAGCCCAGTGT-3′; Probe, 5′-CGGGTCATGGAGCCCAATCC-3′); β-defensin2 (Forward, 5′-CACTCCAGCTGTTGGAAGTTT-3′; Reverse, 5′-GGGTTCTTCTCTGGGAAACA-3′; Probe, 5′-CCTCCTTCTGCCAGGCGTCC-3′); SAA3 (Forward, 5′-GCATCTTGATCCTGGGAGTT-3′; Reverse, 5′-AGACCCTTGACCAGCTTCTTT-3′; Probe, 5′-ACAGCCAAAGATGGGTCCAGTTCA-3′); IL-22RA1 (Forward, 5′-ACATCACCAAGCCACCTGTA-3′; Reverse, 5′-GGTCCAAGACAGGGATCAGT-3′; Probe, 5′-TCCCTGAACGTCCAACGTGTCC-3′). Samples were normalized to GAPDH and displayed as fold induction over vehicle-treated controls unless otherwise stated.

Isolation of Pancreatic Acinar Cells and Leukocytes.

Mice were sacrificed and pancreata removed carefully by trimming fat and mesentery. Dispersed pancreatic acinar cells were isolated using a collagenase digestion method described previously (Menozzi et al. 1990). Isolated primary pancreatic acinar cells were treated in vitro with either rIL-22 or vehicle control (PBS) for up to 4 hours following isolation. Pancreatic leukocytes were prepared with minor modifications of the method developed by Hawkins (Hawkins et al., 1996). Briefly, 3-4 pancreata were pooled together and minced with scissors, then washed twice with buffer A (HBSS+10% FCS). The tissue was resuspended in buffer A containing 2 mg/ml collagenase type IV (Sigma-Aldrich) and incubated in a shaker at 37° C. for 15 min. The suspension was then vortexed at low speed for 20 seconds, centrifuged and the cell pellet was resuspended in red blood cell lysing buffer (Sigma-Aldrich) for 5 min. The cells were spun down, washed 3 times with HBSS+2% BCS and used for surface and intracellular staining

Flow Cytometry.

For surface staining, cells were stained with the following antibodies (Biolegend): AF700 or FITC CD45.2, PE/Cy7-CD3, PE/Cy7-CD19, APC/Cy7-CD4, FITC-CD90, Percp/Cy5.5-CD11b, APC-Sca-1, PB-CD11c, APC/Cy7-NK1.1 and APC-NKp46. For intracellular cytokine staining, immediately after isolation, cells were cultured in RPMI complete medium and stimulated with phorbol myristate acetate (50 ng/ml), ionomycin (1 μg/ml) and brefeldin A (10 μg/ml, eBioscience) for 4 hours. The cells were washed and stained with surface markers. The cells were then fixed and permeabilized using eBioscience kit and following manufacturer's guidelines. PE-IL-22 and isotype control PE-IgG1 (eBioscience) were used for intracellular staining Dead cells were excluded from analysis using violet viability stain (Invitrogen). Flow cytometry data collection was performed on Fortessa LSRII (BD Biosciences) and analyzed using FlowJo software (Tree Star, Inc.).

Statistical Analysis.

Unpaired Student's t-test was used to determine statistical significance and P value of less than 0.05 was considered significant. One way ANOVA plus Tukey's post-hoc test were used to determine the difference among multiple groups, and a P value of less than 0.05 was considered statistically significant. Values are expressed as mean±SEM (Prism 4; GraphPad Software). Unless indicated, results are from at least 3 independent experiments.

### EXAMPLES

The following examples are put forth so as to provide those of ordinary skill in the art with a complete disclosure and description of how to make and use the present invention; they are not intended to limit the scope of what the inventors regard as their invention. Unless indicated otherwise, parts are parts by weight, molecular weight is average molecular weight, temperature is in degrees Centigrade, and pressure is at or near atmospheric.

### Example 1

### Interleukin-22 (IL-22) Induces Phosphorylation of Stat3 and RegIII Genes in the Pancreas

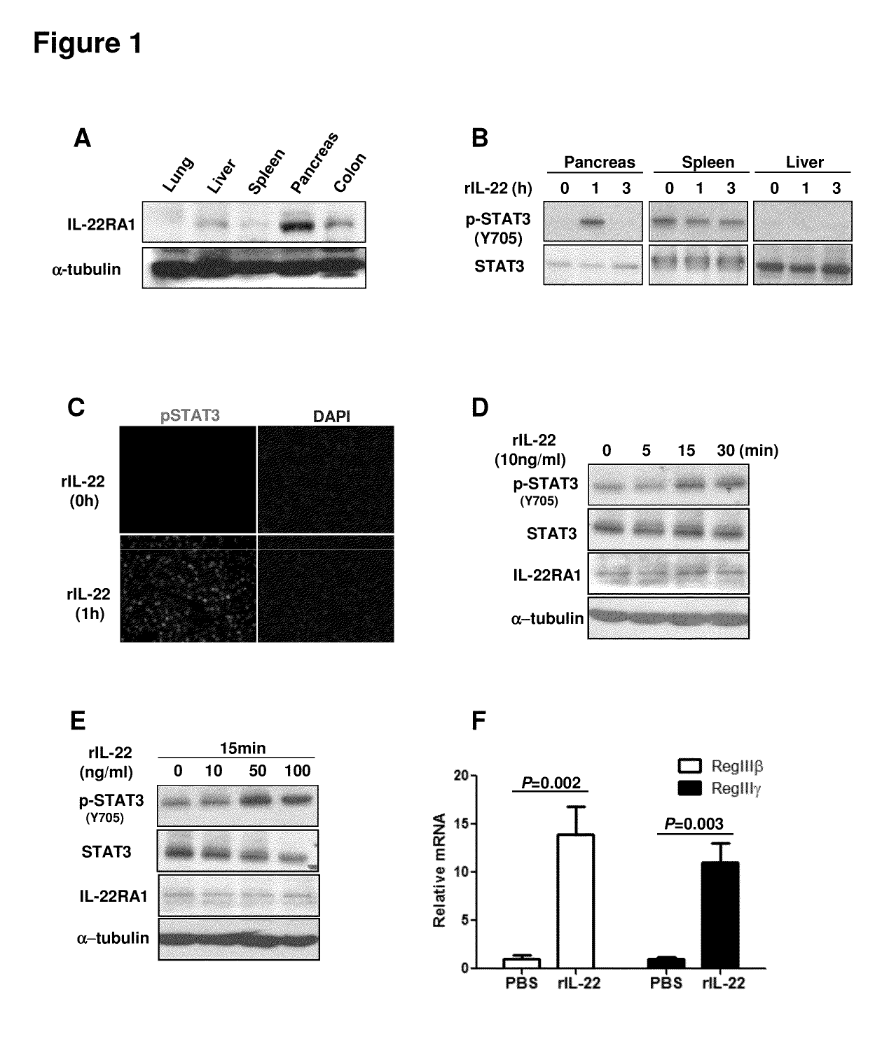

Pancreatic acinar cells express IL-22RA1 mRNA and have been shown to be a target for IL-22 action in vitro (Aggarwal et al., 2001). IL-22RA1 exhibits a restricted expression pattern, with highest level of mRNA expression reported in the pancreas and detectable expression in multiple other tissues, particularly the colon and liver (Gurney et al., 2004). Therefore we first determined the expression of IL-22RA1 in different tissues at a protein level. Compared to the colon and liver, the pancreas has the highest level of IL-22RA1 expression, as shown in FIG. 1A. In contrast to other tissues, the in vivo activation of the IL-22 receptor in the pancreas has not been well defined. To test whether, based on the high expression of IL-22RA1 in the pancreas, the pancreatic tissue would respond strongly to exogenous IL-22, a relatively low dose of rIL-22 (200 ng/mouse and 1 ng/μl) was administered systemically to Balb/c mice, and downstream IL-22 signaling was assessed at specified time points thereafter. Significant STAT3 activation (p-STAT3) was observed in the pancreas at 1 h following exogenous rIL-22 administration as compared to other tissues, see FIG. 1B. Activation of STAT3 in pancreas was also confirmed by immunofluorescence staining, see FIG. 1C. To further confirm acinar cell response, pancreatic acinar cells were isolated and IL-22RA1 receptor activation assessed upon treatment with different doses of rIL-22. As shown, pSTAT3 was induced by rIL-22 in pancreatic acinar cells in a time and dose dependent manner, as illustrated in FIGS. 1D and 1E.

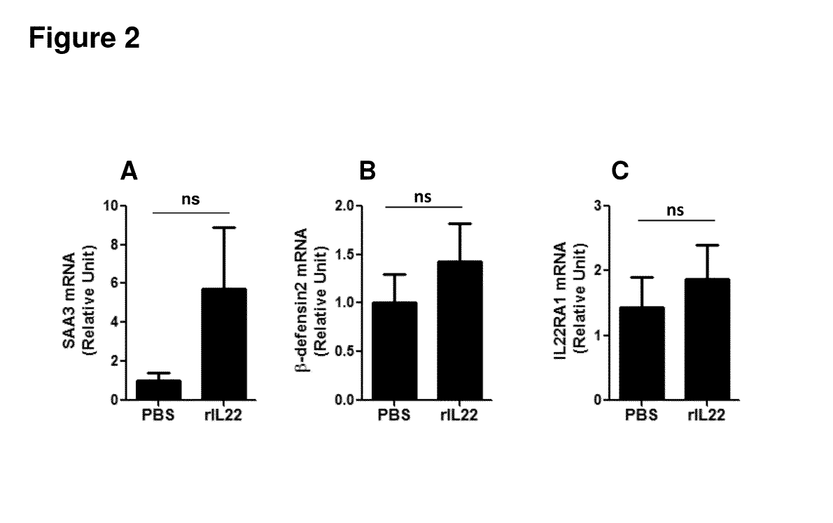

IL-22 has been shown to induce multiple downstream targets in various tissues, such as RegIII genes (also known as pancreatitis-associated protein, PAP), serum amyloid A (SAA), and β-defensins. PAPs are mainly expressed by pancreatic acinar cells and are upregulated during acute pancreatitis (Graf et al., 2002). Emerging evidence supports that PAP proteins play regulatory roles during the inflammatory process in pancreatitis (Gironella et al., 2007; Lin et al., 2008). Such studies demonstrated the protective role of RegIII/PAPs in acute pancreatitis using PAP knockout mice and siRNA knockdown of PAP1 (RegIIIβ) and PAP3 (RegIIIγ)). IL-22 was previously reported to upregulate PAP1 (RegIIIβ) in cultured pancreatic acinar cells (Aggarwal et al., 2001). Therefore, to determine whether exogenous IL-22 administration in vivo can induce the expression of these genes, Balb/c mice were treated with recombinant IL-22 (200 ng/mouse, 1 ng/μl) and their pancreata harvested 24 h later for qPCR analysis. As shown in FIG. 1F, exogenous rIL-22 administration stimulated the expression of RegIIIβ ((PAP1)) and RegIIIγ (PAP3) genes involved in tissue regeneration. However, there was no significant induction in SAA3, β-defensin2, and IL-22RA1, as shown in FIG. 2.

### Example 2

### IL-22RA1 Expression in the Pancreas is Upregulated in Acute Pancreatitis

Since the IL-22RA1 receptor is highly expressed in the pancreas, as demonstrated in FIG. 1A, IL-22RA1 expression during pancreatic inflammation was examined next using two widely accepted independent mouse models of acute pancreatitis (AP): (1) caerulein hyperstimulation, which causes mild to moderate acute pancreatitis (Nakamichi et al., 2005), referred to as ‘caerulein’ in this application; and (2) choline-deficient diet supplemented with DL-ethionine (CDE) feeding which causes severe hemorrhagic acute pancreatitis associated with significant mortality (Habtezion et al., 2011), referred to as ‘CDE diet’ or ‘CDE’ in this application.

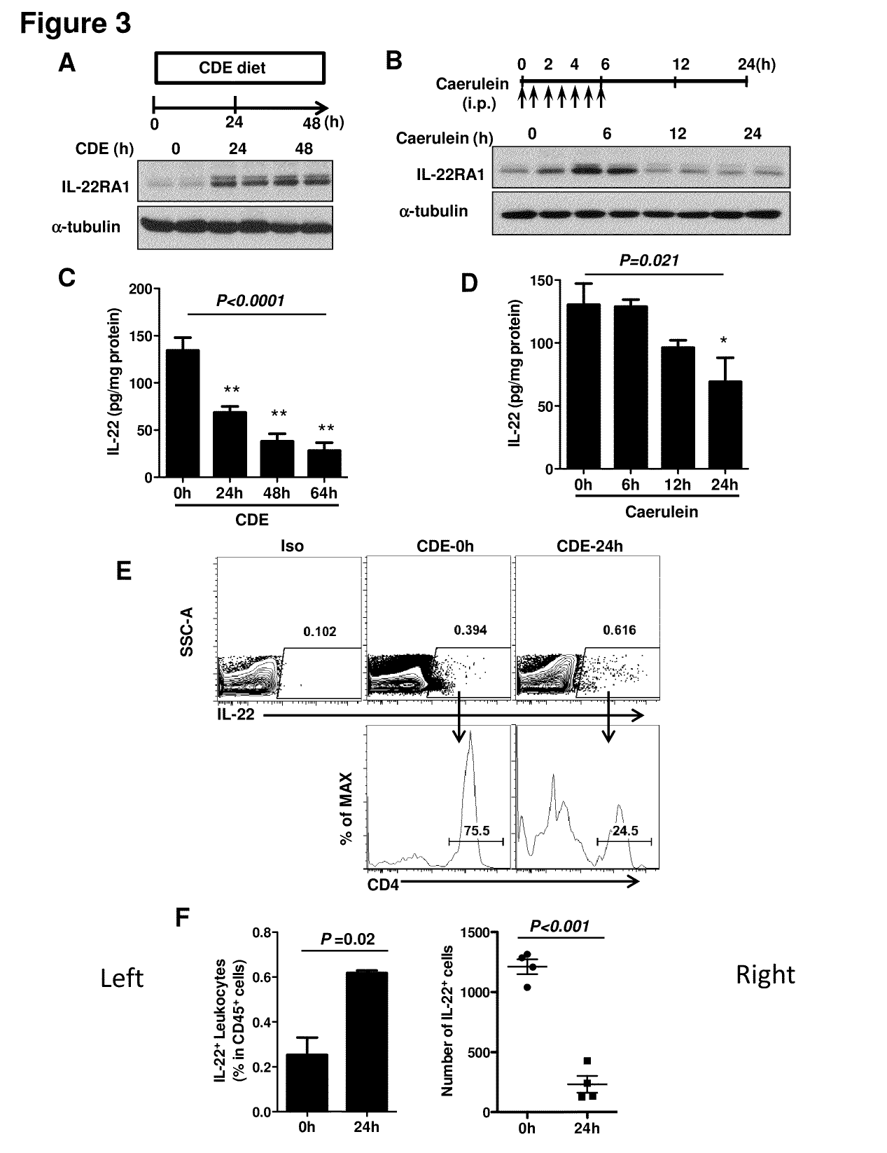

IL-22RA1 expression increased significantly in both models, as illustrated in FIG. 3A for CDE and FIG. 3B for caerulein. Sustained IL-22RA1 expression was noted during the induction and progression of acute pancreatitis in the CDE model, but was reversible during the recovery phase (after the last injection, 6 hours after the first injection of caerulein) in the caerulein model (FIG. 3B). Given the high induction of IL-22RA1 in these acute pancreatitis models, the findings suggest that IL-22RA1 expression is stress-inducible and its activation leads to effects that could be instrumental in attenuating pancreatic injury.

### Example 3

### Pancreatic IL-22 is Reduced During Acute Pancreatitis

To determine the availability of IL-22 in the pancreatic tissue during the disease progression of acute pancreatitis, the expression of IL-22 was assessed over time using the CDE and caerulein mice models of acute pancreatitis. In both models, IL-22 levels decreased significantly over time, as shown in FIG. 3C for the CDE diet model and in FIG. 3D for the caerulein model. Considerable decrease in IL-22 levels was associated with the more severe disease, the CDE diet model, as shown with the CDE feeding over time (FIG. 3C). In contrast, IL-22RA1 expression increased over time, particularly in the CDE diet model, as seen in FIG. 3A. IL-22RA1 expression increased initially in the caerulein model and later decreased, as shown in FIG. 3B.

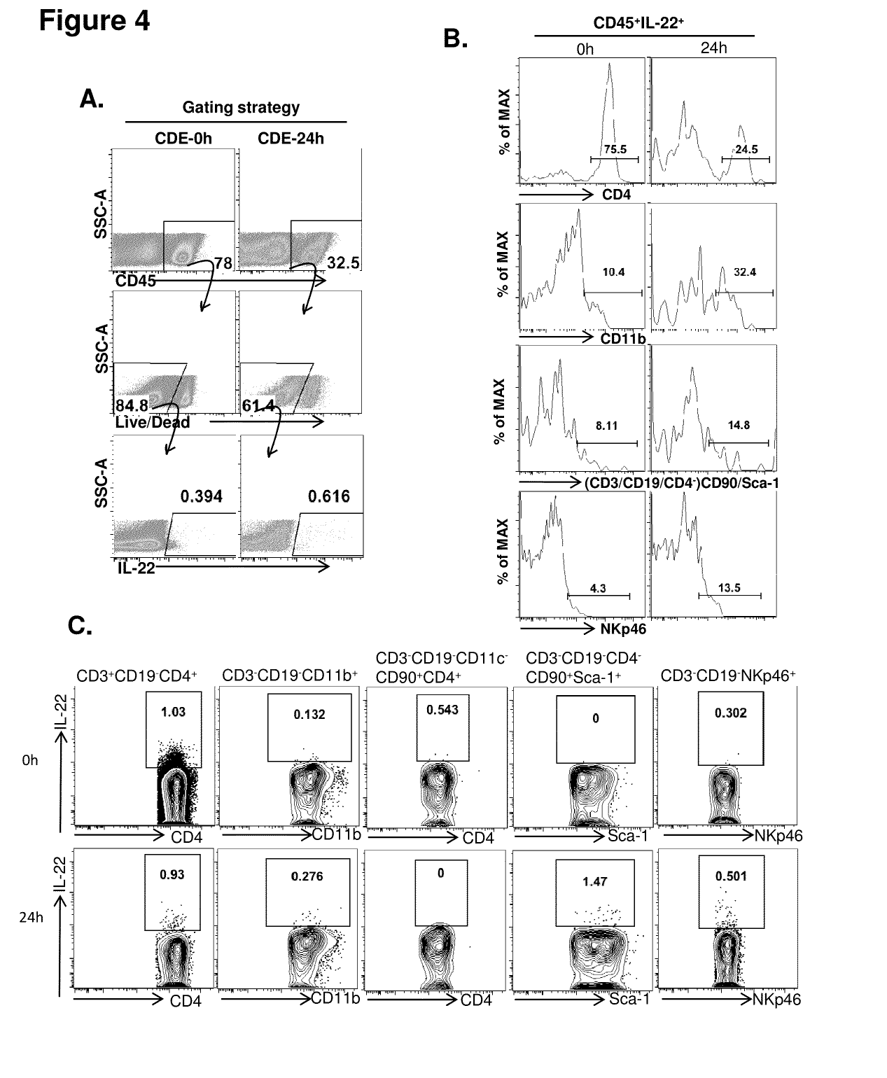

To further assess pancreatic IL-22 expression during acute pancreatitis and to determine the source of the IL-22, pancreatic leukocytes were isolated and subjected to intracellular cytokine staining and flow cytometry analysis. Since pancreatic IL-22 expression, as assessed by ELISA, had declined significantly by 24 h following feeding in the CDE model (as shown in FIG. 3C), the leukocyte IL-22 production at time 0 and 24 h following CDE feeding was compared. Following dead cell exclusion and gating on total (CD45+) leukocytes, it was found that the percent of IL-22+ leukocytes had increased at 24 h following feeding, as shown in FIG. 3E and FIG. 3F. However, since the total number of leukocytes per harvested pancreas had decreased over time (FIG. 4), the absolute number of IL-22+ leukocytes was much lower at 24 h (FIG. 3F, right panel). These results are consistent with the ELISA findings from FIG. 3C suggesting depletion of pancreatic IL-22 during the progression of acute pancreatitis. Under homeostatic conditions, most of the pancreatic IL-22 is produced by CD4+ T cells and during acute pancreatitis IL-22+CD4+ T cells are markedly decreased, as shown in FIG. 3E. Interestingly, using previously described phenotyping strategy (Sonnenberg et al., 2011), the relative increase in percent of IL-22+ leukocytes during acute pancreatitis was in part accounted for by innate immune cells including CD4-LTi (lymphoid tissue inducer) cells and NKp46+ILCs (innate lymphoid cells; FIG. 4). In particular NKp46+ ILCs account for the IL-22+ cells with high fluorescence intensity.

### Example 4

### Exogenous IL-22 Ameliorates Established Acute Pancreatitis

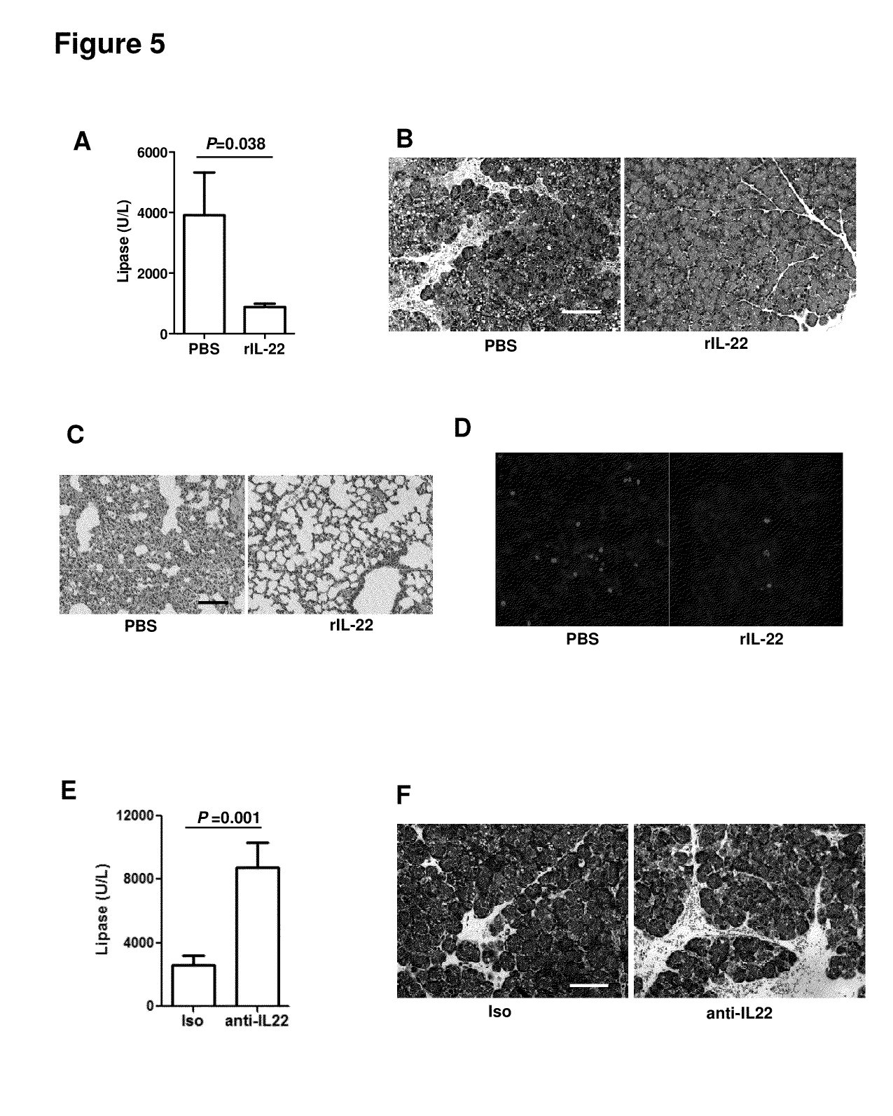

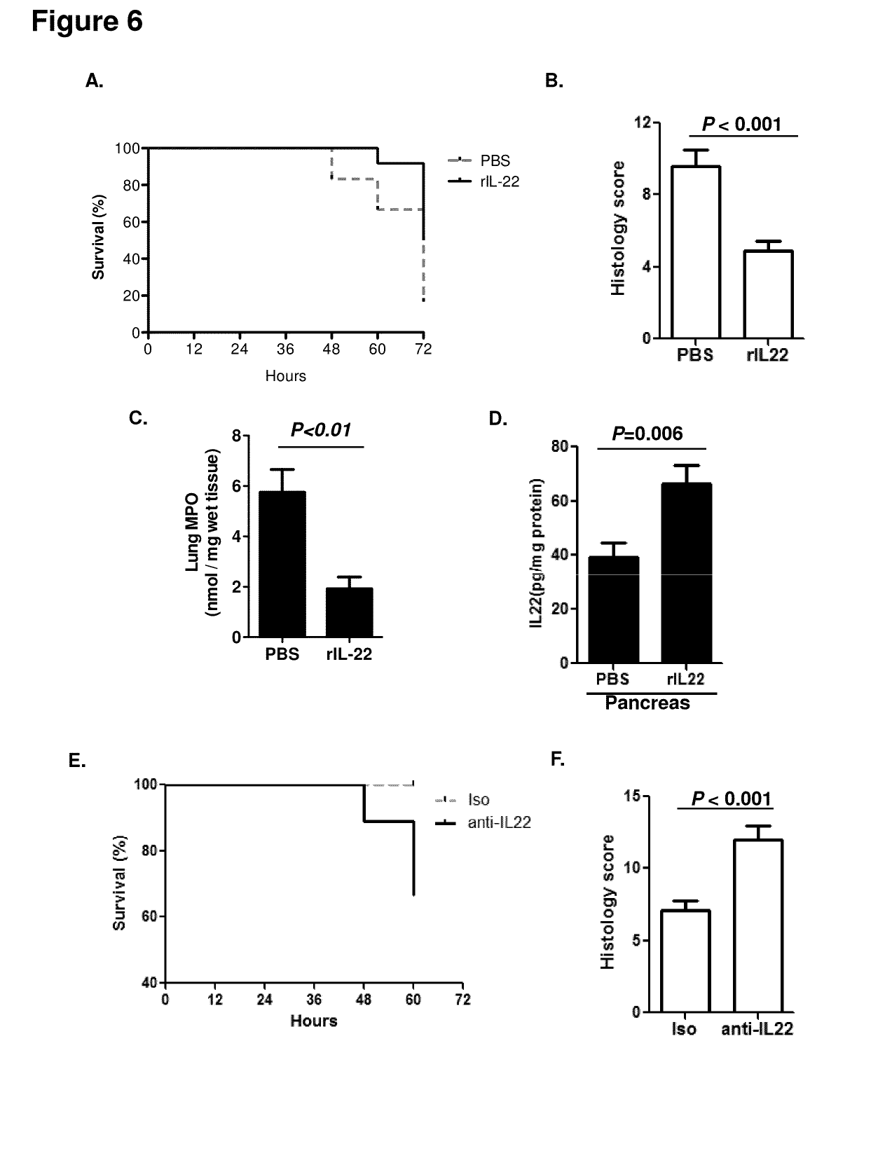

IL-22 expression significantly decreased in the pancreas 12 h and 24 h following the induction of mild to moderate, acute pancreatitis in the mouse model of caerulein hyperstimulation (see FIG. 3D) as well as following the induction of severe hemorrhagic acute pancreatitis in the CDE feeding model (see FIG. 3C). We explored whether supplementation of IL-22 could contribute to attenuation of an established acute pancreatitis. Using the CDE-induced acute pancreatitis model, where we previously had demonstrated a notable pancreatic injury by day 1 (Habtezion et al., 2011), Balb/c mice were administered, 24 h after initiation of the CDE feeding, recombinant IL-22 (200 ng/mouse) or vehicle control (PBS), then serum, pancreas, and lung were harvested at 60 h after the CDE feeding. Serum lipase levels, generally used in the clinical diagnosis of acute pancreatitis, were significantly lower in the group that had received rIL-22 (FIG. 5A), indicating an attenuation of the disease severity. In further support of this observation, histologic examination and blinded scoring of the pancreata indicated a less severe pancreatitis in mice treated with rIL-22 (FIG. 5B and FIG. 6B) than in mice that only received vehicle control. Furthermore, morphologic evidence of lung injury and lung Myeloperoxidase (MPO) levels were significantly reduced by rIL-22 treatment in comparison to vehicle control (PBS) (FIG. 5C and FIG. 6C). Blinded lung histology scores were significantly lower in the rIL-22 treatment group (1.6±0.24; P=0.028) versus the control group (2.8±0.37). In addition rIL-22 treatment group had lower mortality compared with the control group (PBS) (FIG. 6A).

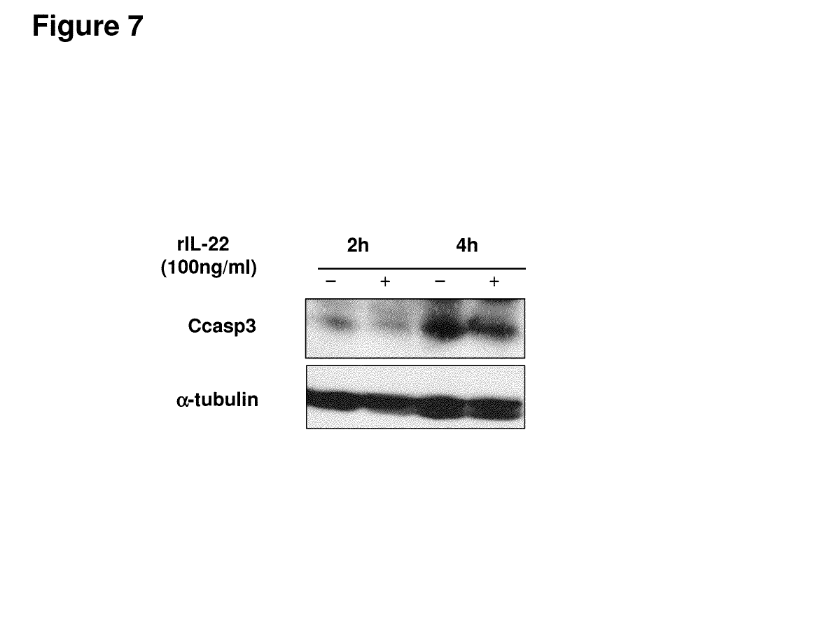

Effective reconstitution of pancreatic IL-22 following systemic administration of recombinant IL-22 was confirmed by ELISA (FIG. 6D). To further determine the role of rIL-22 in the reduction of cell death during acute pancreatitis in vivo, we used TUNEL assay, and observed that rIL-22 treatment significantly reduced apoptosis (FIG. 5D). Similarly, in-vitro treatment of isolated primary pancreatic acinar cells with rIL-22 delayed spontaneous apoptosis (FIG. 7). To further define the role of IL-22 in acute pancreatitis, we treated mice with either isotype control or anti-IL-22 neutralizing mAb. Consistent with the protective role of rIL-22, mice treated with anti-IL-22 mAb had higher mortality and increased serum lipase levels (FIG. 6E and FIG. 5D). In addition, blinded histologic scoring confirmed increased pancreatic injury with blockade of IL-22 (FIG. 5E and FIG. 6F).

### Example 5

### Aryl Hydrocarbon Receptor Inactivation Decreases Pancreatic IL-22 and Worsens Acute Pancreatitis

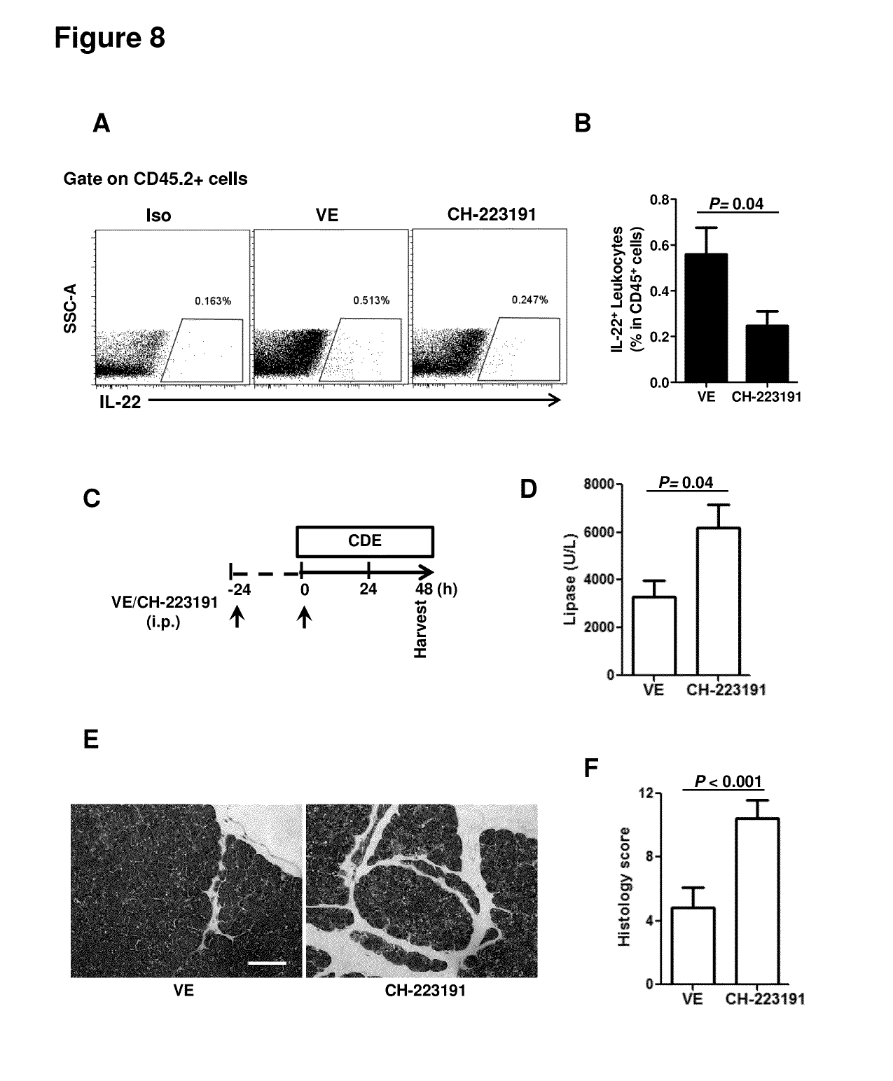

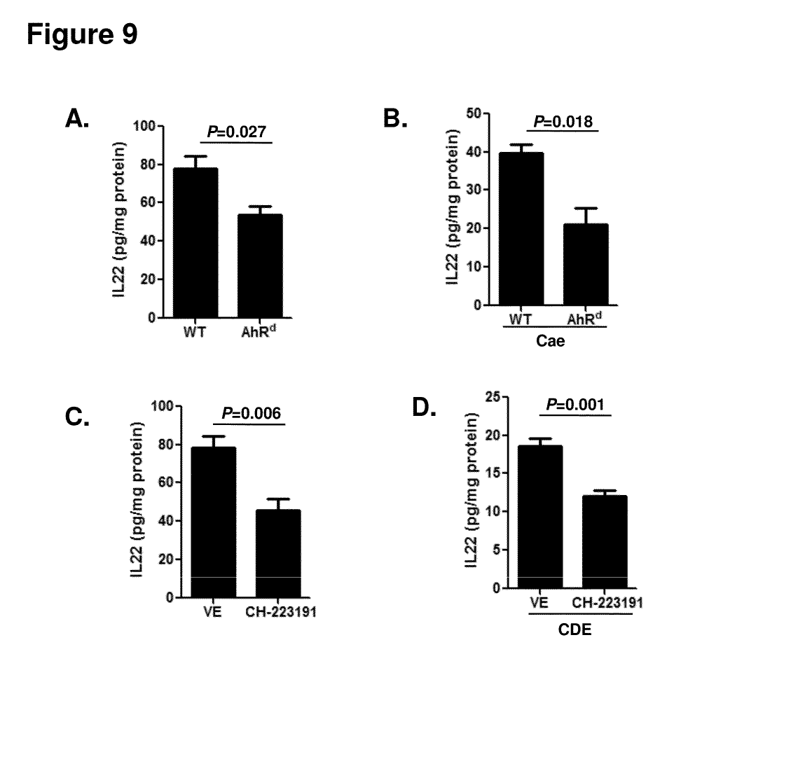

Recent reports show that the transcription factor aryl hydrocarbon receptor (AhR) is required for most leukocytes IL-22 production. Therefore, we investigated whether inhibition of AhR signaling would reduce IL-22 expression in the pancreas and accelerate acute pancreatitis. CH-223191, an AhR antagonist, is a novel chemical compound that has been used widely to inhibit AhR-dependent ligand activation and signaling (Kim et al., 2006). Balb/c mice were treated with either vehicle or CH-223191 (100 μg/mouse) for two consecutive days followed by isolation of pancreatic leukocytes at day 3. The frequency of IL-22-expressing leukocytes in the pancreas was markedly decreased in mice treated with the AhR antagonist in comparison to mice treated with vehicle control, as determined by flow cytometry (FIGS. 8A and 8B) and ELISA (FIG. 9). Furthermore, to determine, if inhibiting AhR signaling would exacerbate acute pancreatitis, we pretreated Balb/c mice with CH-223191, then induced CDE-mediated acute pancreatitis, and analyzed at day 2 (FIG. 8C) due to the increased mortality relative to control treated mice by day 3. Consistent with the AhR requirement for pancreatic IL-22 production and consistent with the observation herein that pancreatic IL-22 exerts a protective role in acute pancreatitis, mice pretreated with AhR antagonist CH-223191 developed a considerably more severe acute pancreatitis when compared to mice treated with vehicle control (FIG. 8D-F).

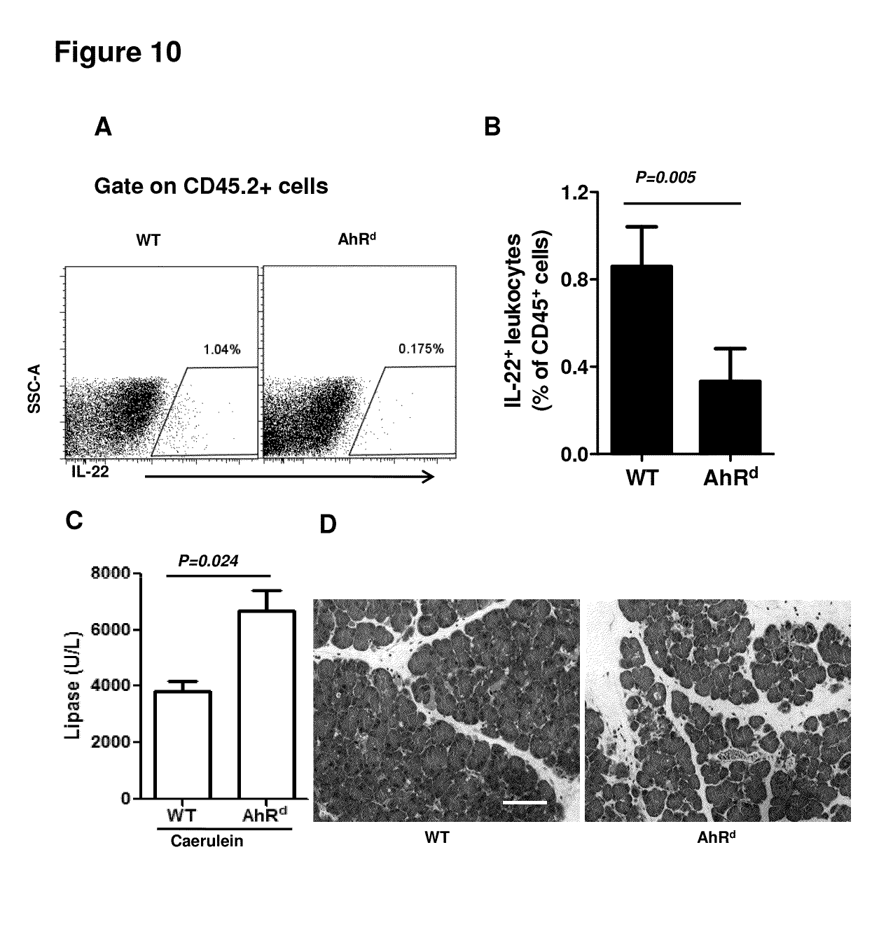

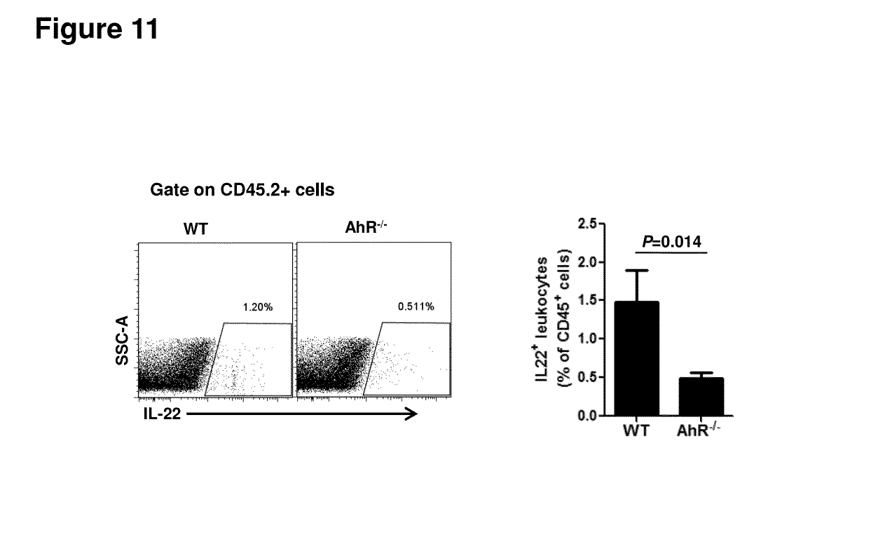

To further delineate and confirm the protective role of AhR in acute pancreatitis, we induced acute pancreatitis in the previously well-described AhR non-responsive (AhR^{d}) mice (Braun et al., 1999) and their wild-type counterparts. The ‘d’ allele codes for AhR protein with reduced affinity for known ligands as a result of mutations in its ligand-binding site. Consistent with the results above, when the AhR antagonist CH-223191 was utilized to inhibit AhR-dependent ligand activation and signaling, pancreatic leukocytes from AhR^{d} mice had reduced levels of IL-22 as compared with those from their wild-type counterparts, as measured by flow cytometry (FIGS. 10A and 10B) and ELISA (FIG. 9). Reduced levels of pancreatic IL-22 were also present in the pancreata of AhR mutant (AhR^{-/-}) (FIG. 11). We next investigated whether AhR^{d} mice, which have a C57BL/6 background, would, due to the decreased expression of IL-22, be more sensitive to the induction of acute pancreatitis. Caerulein hyperstimulation in AhR^{d} mice resulted in a more severe pancreatitis (FIGS. 10C and 10D).

### Example 6

### Aryl Hydrocarbon Receptor Mediates Protective Function of IL-22 in Acute Pancreatitis

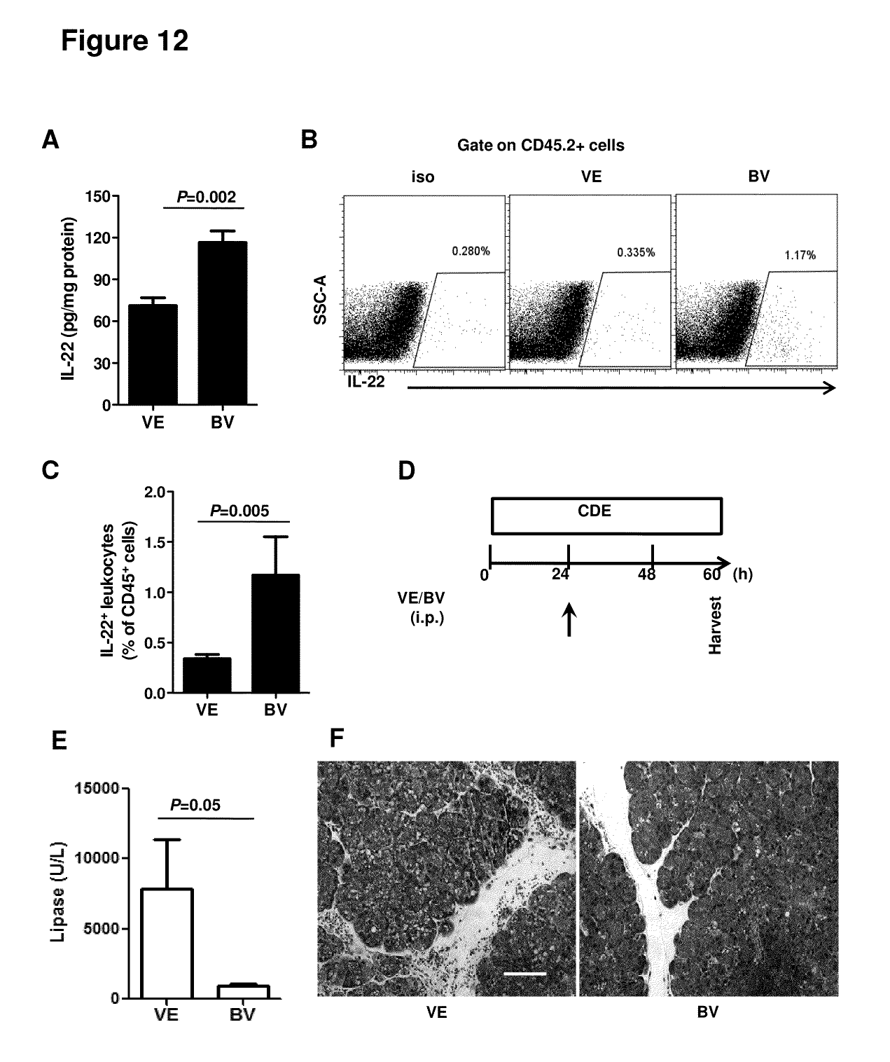

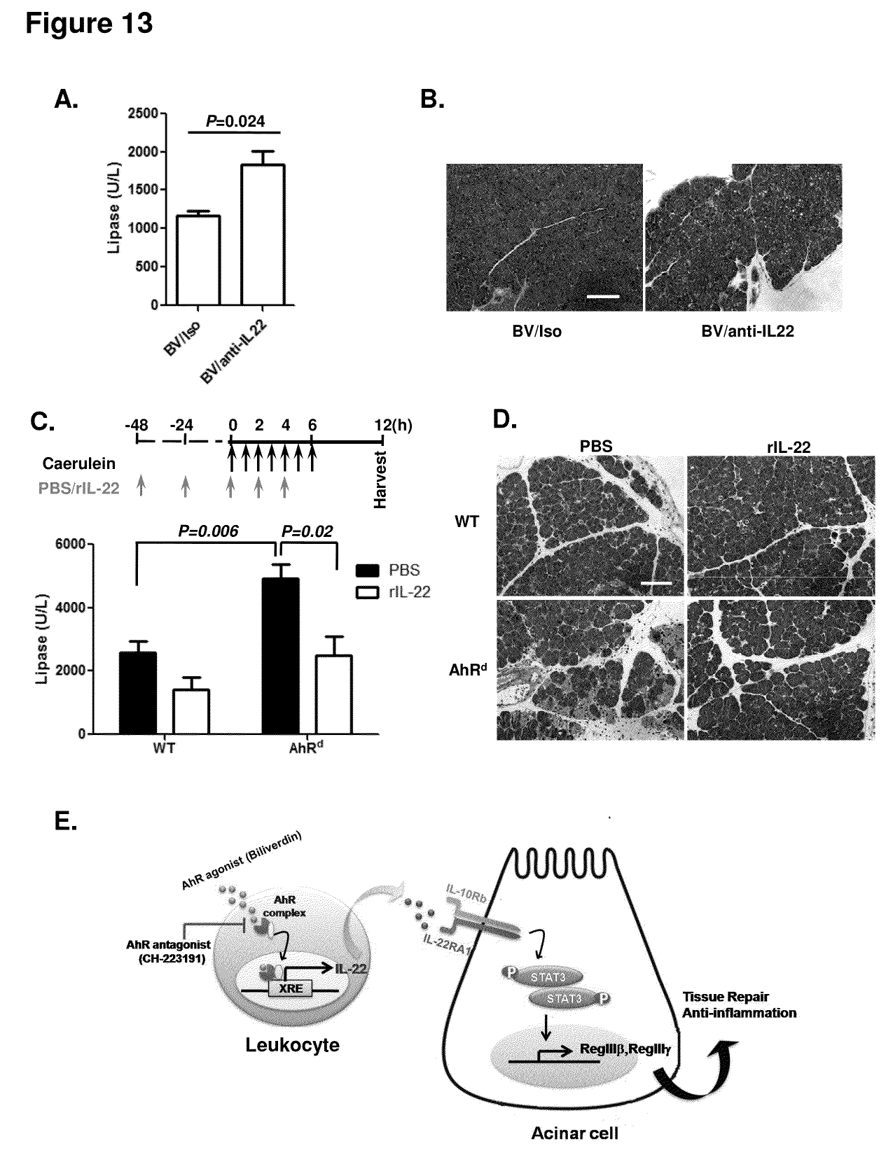

Although environmental toxins remain the ligands that have been characterized the most so far, more and more endogenous aryl hydrocarbon receptor (AhR) ligands such as bilirubin and biliverdin are being discovered and appreciated for a potential therapeutic benefit (Phelan et al., 1998). Based on the studies described in Example 5, where the aryl hydrocarbon receptor was inhibited or deleted, we investigated whether biliverdin, an endogenous AhR ligand, would alter pancreatic IL-22 levels and protect against acute pancreatitis. Indeed, biliverdin administration to mice led to a significant increase in pancreatic IL-22, as measured by ELISA (FIG. 12A) and flow cytometry (FIGS. 12B and 12C). Moreover, mice treated with a single dose of biliverdin (FIG. 12D) were protected against acute pancreatitis, as shown by the serum lipase (FIG. 12E) and histologic assessment (FIG. 12F). To confirm the relationship between AhR activation and IL-22 mediated protection, we conducted IL-22 inhibition experiments, using anti-IL22 antibody, in mice with CDE-mediated acute pancreatitis that were treated with a single dose of biliverdin (BV/anti-IL22 mice). In a group of control mice, an isotype control antibody (Iso) was used instead of the anti-IL22 antibody (BV/Iso mice). Anti-IL-22 in part reversed the protective effect of biliverdin, as shown by the elevated serum lipase values (FIG. 13A) and development of pancreatic injury (FIG. 13B), in comparison to the control mice.

Finally, to further verify the importance of AhR activation in protecting against acute pancreatitis via pancreatic IL-22, we tested whether IL-22 supplementation could render AhR^{d} mice less susceptible to acute pancreatitis. Exogenous IL-22 protected AhR^{d} mice and prevented that these developed acute pancreatitis, as shown by the decrease in serum lipase levels (FIG. 13C) and pancreatic injury (FIG. 13D), in comparison to vehicle control. Taken together, these results show the significance of AhR activation in pancreatic IL-22 induction that allows for a cross talk between immune cells and pancreatic acinar cells to confer protection in acute pancreatitis, as depicted by the schematic diagram (FIG. 13E).

Although the foregoing invention and its embodiments have been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. Accordingly, the preceding merely illustrates the principles of the invention. It will be appreciated that those skilled in the art will be able to devise various arrangements which, although not explicitly described or shown herein, embody the principles of the invention and are included within its spirit and scope.

### REFERENCES

Aggarwal S, Xie M H, Maruoka M, Foster J, Gurney A L. Acinar cells of the pancreas are a target of interleukin-22. J Interferon Cytokine Res 2001; 21:1047-53.
Alam M S, Maekawa Y, Kitamura A, Tanigaki K, Yoshimoto T, Kishihara K, Yasutomo K. Notch signaling drives IL-22 secretion in CD4+ T cells by stimulating the aryl hydrocarbon receptor. Proc Natl Acad Sci USA 2010; 107:5943-8.
Braun L, Kardon T, El Koulali K, Csala M, Mandl J, Banhegyi G. Different induction of gulonolactone oxidase in aromatic hydrocarbon-responsive or -unresponsive mouse strains. FEBS Lett 1999; 463:345-9.
Demols A, Le Moine O, Desalle F, Quertinmont E, Van Laethem J L, Deviere J. CD4(+)T cells play an important role in acute experimental pancreatitis in mice. Gastroenterology 2000; 118:582-90.
Denison M S & Nagy S R. Activation of the aryl hydrocarbon receptor by structurally diverse exogenous and endogenous chemicals. Annu Rev Pharmacol Toxicol 2003; 43:309-34.
Feng D, Park O, Radaeva S, Wang H, Yin S, Kong X, Zheng M, Zakhari S, Kolls J K, Gao B. Interleukin-22 ameliorates cerulein-induced pancreatitis in mice by inhibiting the autophagic pathway. Int J Biol Sci; 8:249-57.
Gaisano H Y, Gorelick F S. New insights into the mechanisms of pancreatitis. Gastroenterology 2009; 136:2040-4.
Gironella M, Folch-Puy E, LeGoffic A, Garcia S, Christa L, Smith A, Tebar L, Hunt S P, Bayne R, Smith A J, Dagorn J C, Closa D, Iovanna J L. Experimental acute pancreatitis in PAP/HIP knock-out mice. Gut 2007; 56:1091-7.
Graf R, Schiesser M, Lussi A, Went P, Scheele G A, Bimmler D. Coordinate regulation of secretory stress proteins (PSP/reg, PAP I, PAP II, and PAP III) in the rat exocrine pancreas during experimental acute pancreatitis. J Surg Res 2002; 105:136-44.
Gravante G, Garcea G, Ong S L, Metcalfe M S, Berry D P, Lloyd D M, Dennison A R. Prediction of Mortality in Acute Pancreatitis: A Systematic Review of the Published Evidence. Pancreatology 2009; 9:601-614.
Gurney A L. IL-22, a Th1 cytokine that targets the pancreas and select other peripheral tissues. Int Immunopharmacol 2004; 4:669-77.
Habtezion A et al. Panhematin provides a therapeutic benefit in experimental pancreatitis. Gut 2011 60:671-9.
Hankinson O, 1995, Annu Rev Pharmacol Toxicol 35:307-340, “The Aryl hydrocarbon receptor complex”).
Hawkins T A, Gala R R, Dunbar J C. The lymphocyte and macrophage profile in the pancreas and spleen of NOD mice: percentage of interleukin-2 and prolactin receptors on immunocompetent cell subsets. J Reprod Immunol 1996; 32:55-71.
Kim S H, Henry E C, Kim D K, Kim Y H, Shin K J, Han M S, Lee T G, Kang J K, Gasiewicz T A, Ryu S H, Suh P G. Novel compound 2-methyl-2H-pyrazole-3-carboxylic acid (2-methyl-4-o tolylazo-phenyl)-amide (CH-223191) prevents 2,3,7,8-TCDD-induced toxicity by antagonizing the aryl hydrocarbon receptor. Mol Pharmacol 2006; 69:1871-8.
Lejeune D, Dumoutier L, Constantinescu S, Kruijer W, Schuring a JJ, Renauld J C. Interleukin-22 (IL-22) activates the JAK/STAT, ERK, JNK, and p38 MAP kinase pathways in a rat hepatoma cell line. Pathways that are shared with and distinct from IL-10. J Biol Chem 2002; 277:33676-82.
Leung P S & Ip S P (2006). Pancreatic acinar cell: Its role in acute pancreatitis. The Intl J Biochem Cell Biol 2006; 38(7):1024-1030.
Leung P S & Chan C C. Role of oxidative stress in pancreatic inflammation. Antiox. Red. Sign. 2009; 11(1):135-165.
Liang S C, Tan X Y, Luxenberg D P, Karim R, Dunussi-Joannopoulos K, Collins M, Fouser L A. Interleukin (IL)-22 and IL-17 are coexpressed by Th17 cells and cooperatively enhance expression of antimicrobial peptides. J Exp Med 2006; 203:2271-9.
Lin Y Y, Viterbo D, Mueller C M, Stanek A E, Smith-Norowitz T, Drew H, Wadgaonkar R, Zenilman M E, Bluth M H. Small-interference RNA gene knockdown of pancreatitis-associated proteins in rat acute pancreatitis. Pancreas 2008; 36:402-10.
Mayerle J. A novel role for leucocytes in determining the severity of acute pancreatitis. Gut 2009; 58:1440-1.
Menozzi D, Jensen R T, Gardner J D. Dispersed pancreatic acinar cells and pancreatic acini. Methods Enzymol 1990; 192:271-9.
Mews P et al. Pancreatic stellate cells respond to inflammatory cytokines potential role in chronic pancreatitis. Gut 2002; 50:535-541.
Monteleone I, Rizzo A, Sarra M, Sica G, Sileri P, Biancone L, MacDonald T T, Pallone F, Monteleone G. Aryl hydrocarbon receptor-induced signals up-regulate IL-22 production and inhibit inflammation in the gastrointestinal tract. Gastroenterology 2011; 141:237-48, 248 el.
Nakamichi I, Habtezion A, Zhong B, Contag C H, Butcher E C, Omary M B. Hemin-activated macrophages home to the pancreas and protect from acute pancreatitis via heme oxygenase-1 induction. J Clin Invest 2005; 115:3007-14.
Nguyen L P & Bradfield C A. The Search for Endogenous Activators of the Aryl Hydrocarbon Receptor. Chem. Res. Toxicol 2008; 21:102-116.
Omary M B, Lugea A, Lowe A W, Pandol S J. The pancreatic stellate cell: a star on the rise in pancreatic diseases. J Clin Invest 2007; 117(1):50-59.
Papachristou G I. Prediction of severe acute pancreatits: Current knowledge and novel insights. World J Gastroenterol 2008; 14(41):6273-6275.
Phelan D, Winter G M, Rogers W J, Lam J C, Denison M S. Activation of the Ah receptor signal transduction pathway by bilirubin and biliverdin. Arch Biochem Biophys 1998; 357:155-63.
Radaeva S, Sun R, Pan H N, Hong F, Gao B. Interleukin 22 (IL-22) plays a protective role in T cell-mediated murine hepatitis: IL-22 is a survival factor for hepatocytes via STAT3 activation. Hepatology 2004; 39:1332-42.
Sonnenberg G F, Fouser L A, Artis D. Border patrol: regulation of immunity, inflammation and tissue homeostasis at barrier surfaces by IL-22. Nat Immunol 2011; 12:383-90.
Sonnenberg G F, Monticelli L A, Elloso M M, Fouser L A, Artis D. CD4(+) lymphoid tissue inducer cells promote innate immunity in the gut. Immunity 2011; 34:122-34.
Werner J, Hartwig H, Uhl W, Mueller C, Buechler M W. Pancreatology 2003; 3:115-127.
Witt H et al. Chronic pancreatitis: challenges and advances in pathogenesis, genetics, diagnosis and therapy. Gastroenterology 2007; 132(4):1557-1573.
Xie M H, Aggarwal S, Ho W H, Foster J, Zhang Z, Stinson J, Wood W I, Goddard A D, Gurney A L. Interleukin (IL)-22, a novel human cytokine that signals through the interferon receptor-related proteins CRF2-4 and IL-22R. J Biol Chem 2000; 275:31335-9.
Xu et al. A soluble class II cytokine receptor, IL-22RA2, is a naturally occurring IL-22 antagonist. Proc. Natl. Acad. Sci. U.S.A., 98 (2001), pp. 9511-9516.
Xue J, Li X, Jiao S, Wei Y, Wu G, Fang J. Prolyl hydroxylase-3 is down-regulated in colorectal cancer cells and inhibits IKKbeta independent of hydroxylase activity. Gastroenterology 2010; 138:606-15.
Zenewicz L A, Yancopoulos G D, Valenzuela D M, Murphy A J, Stevens S, Flavell R A Innate and adaptive interleukin-22 protects mice from inflammatory bowel disease. Immunity 2008; 29:947-57.
Zenewicz L A, Flavell R A. Recent advances in IL-22 biology. Int Immunol 2011; 23:159-63.

What is claimed is:

1. A method of attenuating an acute pancreatic inflammation in a mammalian subject, comprising administering a composition comprising a regulator of pancreatic interleukin-22 (IL-22) expression in a dosage and dosing regimen effective to attenuate pancreatic inflammation and pancreatic tissue damage in said mammalian subject.

2. The method in accordance to claim 1, wherein the regulator of pancreatic IL-22 expression is a modulator of aryl hydrocarbon receptor (AhR).

3. The method in accordance to claim 2, wherein the regulator is an AhR agonist.

4. The method in accordance to claim 1, wherein administering said composition is furthermore effective to attenuate pancreatitis-associated acute lung injury.

5. The method in accordance to claim 1, wherein administering said composition is furthermore effective to prevent pancreatitis-associated acute lung injury.

6. A method of attenuating a pancreatic inflammation in a mammalian subject at risk of developing a chronic pancreatic disorder, comprising administering a composition comprising a regulator of pancreatic IL-22 expression in a dosage and dosing regimen effective to attenuate pancreatic inflammation and pancreatic tissue damage in said mammalian subject.

7. The method in accordance to claim 6, wherein the regulator of pancreatic interleukin-22 expression is a modulator of AhR.

8. The method in accordance to claim 7, wherein the regulator is an AhR agonist.

9. A method of attenuating acute lung injury that is associated with pancreatic inflammation in a mammalian subject, comprising administering a composition comprising a regulator of pancreatic IL-22 expression in a dosage and dosing regimen effective to attenuate said acute lung injury that is associated with pancreatic inflammation in said mammalian subject.

10. The method in accordance to claim 9, wherein the regulator of pancreatic interleukin-22 expression is a modulator of AhR.

11. The method in accordance to claim 10, wherein the regulator is an AhR agonist.


SEQUENCE LISTING (15 sequences)

SEQ ID NO: 1 - DNA, 21 bp, Artificial Sequence
  Primer
tggaagacagacaagatgctg

SEQ ID NO: 2 - DNA, 20 bp, Artificial Sequence
  Primer
taagagcatcaggcaggaga

SEQ ID NO: 3 - DNA, 21 bp, Artificial Sequence
  Primer
cctccaacagcctgctccgtc

SEQ ID NO: 4 - DNA, 20 bp, Artificial Sequence
  Primer
tcctgtcctccatgatcaaa

SEQ ID NO: 5 - DNA, 19 bp, Artificial Sequence
  Primer
tgggttcatagcccagtgt

SEQ ID NO: 6 - DNA, 20 bp, Artificial Sequence
  Primer
cgggtcatggagcccaatcc

SEQ ID NO: 7 - DNA, 21 bp, Artificial Sequence
  Primer
cactccagctgttggaagttt

SEQ ID NO: 8 - DNA, 20 bp, Artificial Sequence
  Primer
gggttcttctctgggaaaca

SEQ ID NO: 9 - DNA, 20 bp, Artificial Sequence
  Primer
cctccttctgccaggcgtcc

SEQ ID NO: 10 - DNA, 20 bp, Artificial Sequence
  Primer
gcatcttgatcctgggagtt

SEQ ID NO: 11 - DNA, 21 bp, Artificial Sequence
  Primer
agacccttgaccagcttcttt

SEQ ID NO: 12 - DNA, 24 bp, Artificial Sequence
  Primer
acagccaaagatgggtccagttca

SEQ ID NO: 13 - DNA, 20 bp, Artificial Sequence
  Primer
acatcaccaagccacctgta

SEQ ID NO: 14 - DNA, 20 bp, Artificial Sequence
  Primer
ggtccaagacagggatcagt

SEQ ID NO: 15 - DNA, 22 bp, Artificial Sequence
  Primer
tccctgaacgtccaacgtgtcc